(19) United States
(12) Patent Application Publication
(10) Pub. No.: US 2022/0380813 A1
(43) Pub. Date: Dec. 1, 2022

(54) PROCESS FOR PRODUCING ETHANOL

(71) Applicant: DSM IP ASSETS B.V., Heerlen (NL)

(72) Inventors: Ingrid Maria VUGT-VAN LUTZ, Echt (NL); Hans Marinus Charles Johannes DE BRUIJN, Echt (NL); Rolf POLDERMANS, Echt (NL)

(21) Appl. No.: 17/774,361

(22) PCT Filed: Nov. 9, 2020
(86) PCT No.: PCT/EP2020/081529

§ 371 Date: May 4, 2022

(30) Foreign Application Priority Data
Nov. 8, 2019 (EP) 19207963.0

(51) Int. Cl. C12P 7/06; C12N 1/18; C12N 15/52

(52) U.S. Cl. CPC C12P 7/06; C12N 1/185; C12Y 503/01003; C12Y 207/01006; C12Y 101/01006; C12Y 602/01001; C12N 15/52; C12R 2001/865

(57) ABSTRACT

The invention relates to a process for the production of ethanol, the process comprising fermenting of a carbon source composition with a recombinant yeast,

wherein the carbon source composition comprises at least glucose and arabinose; and
wherein the recombinant yeast comprises arabinose isomerase activity, ribulokinase activity, ribulose phosphate epimerase activity, glycerol uptake activity and glycerol conversion capacity; and
wherein the recombinant yeast further comprises a genetic modification leading to the reduction, downregulation, inhibition and/or elimination of the activity of a homologous protein with glycerol-efflux activity; and
wherein each of the glucose and the arabinose is converted into ethanol.

In addition, the invention relates to a recombinant yeast that can be used in such a process.

### FIELD OF THE INVENTION

The invention relates to a process for producing ethanol.

### BACKGROUND OF THE INVENTION

Bioethanol is a sustainable way to supplement or even replace fossil-based transport fuels because it combines a lower carbon footprint with compatibility with current internal combustion engine technology.

A distinction is made between first generation bioethanol and 1.5 or second generation bioethanol. So-called first generation industrial bioethanol production processes are for example based on fermentation of hydrolysed corn starch or sugar-cane sucrose. Glucose, a hexose, is the main sugar product resulting from the hydrolysis of such corn starch or sugar-cane sucrose. This glucose can subsequently be fermented with a yeast to produce ethanol.

So-called 1.5 or second generation bioethanol can be produced from cellulose and/or hemicellulose containing biomass. Especially the hydrolysis of fractions of plant biomass is economically attractive. Fractions of plant biomass may be hydrolyzed into a hydrolysate comprising different types of free monomeric sugars, including hexoses and/or pentoses. An example of such a hexose (i.e. a six-carbon sugar) is glucose. Examples of pentoses (i.e. five-carbon sugars) include xylose and arabinose. In addition, such hydrolysates may contain glycerol and/or acetic acid.

A major challenge in second generation ethanol production is the fermentation of the mixed carbon sources (such as glucose, arabinose, xylose, glycerol and/or acetic acid) included in such a hydrolysate.

Yeasts are the organisms of choice in the ethanol industry, but for example the commonly used Saccharomyces cerevisiae cannot utilize five-carbon (C5) sugars contained in the hemicellulose component of biomass feedstocks. Hemicellulose can make up to 20-30% of biomass, with xylose and arabinose being the most abundant five-carbon sugars. Yeasts can be genetically modified to increase capability to ferment for example five-carbon sugars, but the conversion of a mixed carbon source composition requires multiple genetic modifications where one modification may influence another and vice versa. In addition, the rates of conversion may differ for each different carbon source.

Another major challenge is the aspect of glucose-repression. Glucose repression refers to the phenomenon that yeast cells grown on glucose repress the expression of a number of genes that are required for the metabolism of alternate carbon sources. In prior art fermentation of mixed sugar compositions, yeasts preferentially utilize glucose, while xylose and arabinose are only used at the moment when glucose is nearly completely depleted.

U.S. Pat. No. 9,551,015 describes the phenomenon of glucose-repression and a process for the production of a fermentation product, such as ethanol, from a sugar composition comprising glucose, galactose and arabinose. The described process comprises: a) fermenting said sugar composition in the presence of a recombinant yeast belonging to the genera Saccharomyces, Kluyveromyces, Candida, Pichia, Schizosaccharomyces, Hansenula, Kloeckera, Schwanniomyces and/or Yarrowia, and b) recovering the fermentation product, wherein said recombinant yeast comprises gene araA, araB and araD, wherein each of said glucose, galactose and arabinose is converted into at least one fermentation product, such as ethanol.

Although commercially interesting results are obtained with the yeast cell and process described in U.S. Pat. No. 9,551,015, it would be desirable to have a quicker and/or more complete conversion of a pentose sugar such as arabinose. It may further be desirable to also convert carbon sources other than the mentioned sugars, such as for example glycerol and/or acetic acid or any acetate salt thereof.

WO2015/028583 describes a yeast cell that is genetically modified comprising: a) one or more nucleic acid sequence encoding a glycerol dehydrogenase (E.C. 1.1.1.6); b) one or more nucleic acid sequence encoding a dihydroxyacetone kinase (E.C. 2.7.1.28 or E.C. 2.7.1.29) and c) one or more nucleic acid sequence encoding a glycerol transporter. In addition, the cell may comprise one or more nucleic acid sequences encoding a NAD+-dependent acetylating acetaldehyde dehydrogenase. WO2015/028583 further describes a process comprising the preparation of a fermentation product from acetate and from a fermentable carbohydrate—in particular a carbohydrate selected from the group of glucose, fructose, sucrose, maltose, xylose, arabinose, galactose and mannose—which preparation is carried out under anaerobic conditions using the above yeast cell.

WO2015/028583 explains that as acetic acid is often considered to be the most toxic compound present in hydrolysates, there is a desire to further decrease the acetate (acetic acid) concentration in hydrolysates. It is mentioned that one way of increasing the anaerobic acetate conversion potential of the yeast is by introducing a glycerol conversion pathway that for example converts externally added glycerol forcing the yeast cell to convert more acetic acid in order to maintain the redox balance.

In the examples and Table 11, WO2015/028583 illustrates that especially transformant T5, including a glycerol transporter STL1 originating from Zygosaccharomycs rouxii, resulted in the conversion of more glycerol, relative to the reference strain. Also more acetic acid was consumed. The ethanol titer, however, was not the highest in case of this T5, because not all sugars were consumed. Hence, although good results are obtained with the yeast cell and process described in WO2015/028583, there is still room for further improvement, for example in the conversion of pentoses such as xylose and/or arabinose.

Kyung Ok Yu et al, in their article titled “Reduction of glycerol production to improve ethanol yield in an engineered Saccharomyces cerevisiae using glycerol as a substrate”, published in the Journal of Biotechnology vol. 150 (2010), pages 209-214, describe a genetically modified yeast strain. To improve the rate of ethanol production, native glycerol dehydrogenase and dihydroxy-acetone kinase are overexpressed and the genes FPS1 and GPD2 are deleted. The strains were tested on glucose only and the applicability of the strains for the conversion of mixed carbon sources, for example also comprising a pentose, glycerol and/or acetic acid or acetate, was not investigated.

WO2015/023989 describes a recombinant micro-organism comprising as a component a) one or more native and/or heterologous proteins that function to import glycerol into the recombinant micro-organism, where said one or more native and/or heterologous proteins is activated, upregulated or overexpressed; and as a component b) one or more native and/or heterologous enzymes that function in one or more engineered metabolic pathways to convert a carbohydrate source to an alcohol, wherein said one or more native and/or heterologous enzymes is activated upregulated, overexpressed or downregulated.

As an example of component a) the description of WO2015/023989 mentions S. cerevisiae glycerol active transporters and, in passing, some heterologous transporters from other yeast such as C. albicans, Saccharomyces paradoxus and Pichia sorbitophila. For component b) several different alternatives are mentioned in the text.

WO2015/0023989 mentions that the recombinant microorganism may further comprise one or more native and/or heterologous proteins that function to export glycerol from the micro-organism, wherein said one or more native and/or heterologous enzymes that function to export glycerol can be activated, upregulated or downregulated. The description mentions FPS1 as an example of such an enzyme. However, the activation, upregulation or downregulation of FPS1 is not exemplified in the working examples and its functioning not explained.

The exemplified genetically modified strains in WO2015/023989 were tested on glucose and mixtures of glucose, sucrose and fructose (i.e. on cane syrup and cane molasses). FIG. 17B further illustrates a decrease in glycerol for one of the genetically modified strains, but the viability for this genetically modified strain appears decreased as illustrated in FIG. 17C. The applicability of the strain(s) for the conversion of mixed carbon feedstocks, for example also comprising a pentose and/or acetic acid or acetate, was not investigated.

It would be an advancement in the art to provide a yeast cell and/or process for producing an ethanol, that allows for the conversion of a mixed carbon source composition, including glucose and arabinose and optionally non-sugar carbon sources such as glycerol and/or acetic acid or a salt thereof. It would also be an advancement in the art to provide a yeast cell and/or a process, that allows for an increase in amount and/or rate of arabinose conversion in the presence of glucose. It would further be an advancement in the art to provide a yeast cell, that can be used in the conversion of such a mixed carbon source composition, that still has a commercially interesting viability.

### SUMMARY OF THE INVENTION

It has now surprisingly been found that a mixed carbon source composition can be converted in a satisfactory manner with a process according to the invention.

The invention therefore provides a process for the production of ethanol, the process comprising:

fermenting of a carbon source composition with a recombinant yeast,
wherein the carbon source composition comprises at least glucose and arabinose; and
wherein the recombinant yeast comprises arabinose isomerase activity, ribulokinase activity, ribulose phosphate epimerase activity, glycerol uptake activity and glycerol conversion capacity; and
wherein the recombinant yeast further comprises a genetic modification leading to the reduction, downregulation, inhibition and/or elimination of the activity of a homologous protein with glycerol-efflux activity.
Suitably each of the glucose and the arabinose can be converted into ethanol. Further the process is suitably carried out under anaerobic conditions.

The above process surprisingly allows for an increase in amount and/or rate of arabinose conversion in the presence of glucose. This advantageously may allow one to convert a mixed carbon source composition, including glucose and arabinose, and optionally non-sugar carbon sources such as glycerol and/or acetic acid or a salt thereof.

In addition the present invention provides a recombinant yeast comprising: a gene encoding a heterologous protein having arabinose isomerase activity; a gene encoding a heterologous protein having ribulokinase activity; a gene encoding a heterologous protein having ribulose phosphate epimerase activity; a gene encoding a heterologous protein having glycerol uptake activity; a gene encoding a heterologous protein having NAD+-dependent glycerol dehydrogenase activity; and a genetic modification leading to the reduction, downregulation, inhibition and/or elimination of the activity of a homologous protein with glycerol-efflux activity.

Surprisingly the recombinant yeast can be used in the conversion of a mixed carbon source composition, whilst still maintaining a commercially interesting viability.

As illustrated in the examples, the recombinant yeast and process according to the invention advantageously avoid glucose repression. They advantageously allow the conversion of each of several carbon sources, including hexoses such as glucose, pentoses such as arabinose and/or xylose, glycerol and acetic acid or a salt thereof.

It is further believed that, even under anaerobic conditions, the recombinant yeast and process according to the invention may advantageously allow for the co-conversion of a hexose such as glucose and a pentose such as arabinose and/or xylose.

### BRIEF DESCRIPTION OF SEQUENCE LISTINGS

SEQ ID NO:1 Amino acid sequence of the glycerol transporter from S. pombe;

SEQ ID NO:2 Amino acid sequence of the glycerol transporter from Plasmodium falciparum;

SEQ ID NO:3 Amino acid sequence of the glycerol transporter from Danio rerio;

SEQ ID NO:4 Amino acid sequence of the glycerol transporter from Xenopus tropicalis;

SEQ ID NO:5 Amino acid sequence of the glycerol transporter from Z. rouxii;

SEQ ID NO:6 Amino acid sequence of the acetylating acetaldehyde dehydrogenase (adhE) from E. coli;

SEQ ID NO:7 Amino acid sequence of the glycerol dehydrogenase gldA from E. coli;

SEQ ID NO:8 Amino acid sequence of the dihydroxy acetone DAK1 from S. cerevisiae;

SEQ ID NO:9 Amino acid sequence of the dihydroxy acetone DAK2 from S. cerevisiae;

SEQ ID NO:10 Amino acid sequence of the arabinose isomerase (araA) from L. plantarum;

SEQ ID NO:11 Amino acid sequence of the L-ribulokinase (araB) from L. plantarum;

SEQ ID NO:12 Amino acid sequence of the L-ribulose-5-P-4-epimerase (araD) from L. plantarum;

SEQ ID NO:13 Amino acid sequence of ethanolamine utilizing protein (Ec_eutE) from E. coli;

SEQ ID NO:14 Amino acid sequence of the FPS1 aquaglyceroporin from S. cerevisiae.

In the context of this patent application, each of the above protein/amino acid sequences is preferably encoded by a DNA/nucleic acid sequence that is codon-pair optimized for expression in S. cerevisiae. Such optimization may be carried out by methods well known by a person skilled in the art.

In order to reach an optimal expression one or more promoters may be added. Promoters may be regulated from strong to weak and may include one or more of TDH3, FBA1, ENO2, PGK1, TEF1, HTA1, HHF2, RPL8A, CHO1, RPS3, EFT2, HTA2, ACT1, PFY1, CUP1, ZUO1, VMA6 and/or ANB1, HEM13, YHK8, FET4, TIR4, AAC3.

### DETAILED DESCRIPTION OF THE INVENTION

Throughout the present specification and the accompanying claims, the words “comprise” and “include” and variations such as “comprises”, “comprising”, “includes” and “including” are to be interpreted inclusively. That is, these words are intended to convey the possible inclusion of other elements or integers not specifically recited, where the context allows.

The articles “a” and “an” are used herein to refer to one or to more than one (i.e. to one or at least one) of the grammatical object of the article. By way of example, “an element” may mean one element or more than one element. When referring to a noun (e.g. a compound, an additive, etc.) in the singular, the plural is meant to be included. Thus, when referring to a specific moiety, e.g. “gene”, this means “at least one” of that gene, e.g. “at least one gene”, unless specified otherwise.

When referring to a compound of which several isomers exist (e.g. a D and an L enantiomer), the compound in principle includes all enantiomers, diastereomers and cis/trans isomers of that compound that may be used in the particular aspect of the invention; in particular when referring to such as compound, it includes the natural isomer(s).

The term “carbon source” refers to a source of carbon, preferably a compound or molecule comprising carbon. Suitably the carbon source may be selected from the group consisting of mono-, di- and/or polysaccharides, polyols, acids and acid salts. More preferably the carbon source is a compound selected from the group of glucose, arabinose, xylose, galactose, mannose, rhamnose, fructose, glycerol and acetic acid or a salt thereof. Preferably the carbon source is a carbohydrate.

The term “carbohydrate” is understood herein to be an organic compound made of carbon, oxygen and hydrogen. Suitably the carbohydrate may be selected from the group consisting of mono-, di- and/or polysaccharides, polyols, acids and acid salts. More preferably the carbohydrate is a compound selected from the group of glucose, arabinose, xylose, galactose, mannose, rhamnose, fructose, glycerol, sugar alcohols and acetic acid or a salt thereof.

The term “ferment”, and variations thereof such as “fermenting”, “fermentation” and/or “fermentative”, is used herein in a classical sense, i.e. to indicate that a process is or has been carried out under anaerobic conditions. Anaerobic conditions are herein defined as conditions without any oxygen or in which essentially no oxygen is consumed by the recombinant cell, in particular a recombinant yeast cell, and suitably corresponds to an oxygen consumption of less than 5 mmol/l·h⁻¹, in particular to an oxygen consumption of less than 2.5 mmol/l·h⁻¹, or less than 1 mmol/l·h⁻¹. More preferably 0 mmol/L/h is consumed (i.e. oxygen consumption is not detectable. This suitably corresponds to a dissolved oxygen concentration in a culture broth of less than 5% of air saturation, more suitably to a dissolved oxygen concentration of less than 1% of air saturation, or less than 0.2% of air saturation.

The term “consumption” and variations thereof such as “consuming” and “consume” is used herein to indicate that a certain feed or feed component is “converted”, “utilized” or “used up”. For example, if a certain sugar is “consumed” by a yeast, it is hence “used up”, i.e. converted by the yeast. When certain feed components, e.g. glucose and arabinose, are “co-consumed”, this may refer to a situation where at least part of one of such components, e.g. arabinose, is (at least partially) converted, whilst at the same time also at least part of the other component, e.g. glucose, is (at least) converted.

By the terms “conversion”, “converting”, “convert” and/or “converted” is herein understood that one compound is (being) changed into another compound. Such conversion may occur intracellular or extracellular.

By the phrases “co-conversion”, “co-converted” and/or “co-converting” is herein understood that at least part of one compound is being converted (i.e. being changed) whilst at the same time also at least part of another compound is being converted. When certain feed components, e.g. glucose and arabinose, are “co-converted”, this may refer to a situation where at least part of one of such components, e.g. arabinose, is (at least partially) converted, whilst at the same time also at least part of the other component, e.g. glucose, is (at least) converted. Preferably such co-conversion occurs simultaneously.

The term “cell” refers to a eukaryotic or prokaryotic organism, preferably occurring as a single cell. In the present invention the recombinant cell is a recombinant yeast cell. That is, the recombinant cell is selected from the group of genera consisting of yeast.

The terms “yeast” and “yeast cell” are used herein interchangeably and refer to a phylogenetically diverse group of single-celled fungi, most of which are in the division of Ascomycota and Basidiomycota. The budding yeasts (“true yeasts”) are classified in the order Saccharomycetales. Most preferably the recombinant yeast is a recombinant yeast cell derived from the genus of Saccharomyces. Still more preferably the recombinant yeast or recombinant yeast cell is derived from a yeast cell of the species Saccharomyces cerevisiae.

The term “recombinant”, for example referring to a “recombinant yeast”, a “recombinant cell”, “recombinant micro-organism” and/or “recombinant strain” as used herein, refers to a yeast, cell, micro-organism or strain, respectively, containing nucleic acid which is the result of one or more genetic modifications. Simply put the yeast, cell, micro-organism or strain contains a different combination of nucleic acid from (either of) its parent(s). To construe a recombinant yeast, cell, micro-organism or strain recombinant DNA technique(s) and/or another mutagenic technique(s) can be used. In particular a recombinant yeast and/or a recombinant yeast cell may comprise nucleic acid not present in the corresponding wild-type yeast and/or cell, which nucleic acid has been introduced into that yeast and/or yeast cell using recombinant DNA techniques (i.e. a transgenic yeast and/or cell), or which nucleic acid not present in said wild-type yeast and/or cell is the result of one or more mutations—for example using recombinant DNA techniques or another mutagenesis technique such as UV-irradiation—in a nucleic acid sequence present in said wild-type yeast and/or yeast cell (such as a gene encoding a wild-type polypeptide) or wherein the nucleic acid sequence of a gene has been modified to target the polypeptide product (encoding it) towards another cellular compartment. Further, the term “recombinant” may suitably relate to a yeast, cell, micro-organism or strain from which nucleic acid sequences have been removed, for example using recombinant DNA techniques.

By a recombinant yeast comprising or having a certain activity is herein understood that the recombinant yeast may comprise one or more nucleic acid sequences encoding for a protein having such activity. Hence allowing the recombinant yeast to functionally express such a protein.

The term “transgenic” as used herein, for example referring to a “transgenic yeast” and/or a “transgenic cell”, refers to a yeast and/or cell, respectively, containing nucleic acid not naturally occurring in that yeast and/or cell and which has been introduced into that yeast and/or cell using recombinant DNA techniques, such as a recombinant yeast and/or cell.

The term “mutated” as used herein regarding proteins or polypeptides means that at least one amino acid in the wild-type or naturally occurring protein or polypeptide sequence has been replaced with a different amino acid, inserted or deleted from the sequence via mutagenesis of nucleic acids encoding these amino acids. Mutagenesis is a well-known method in the art, and includes, for example, site-directed mutagenesis by means of PCR or via oligonucleotide-mediated mutagenesis as described in Sambrook et al., Molecular Cloning-A Laboratory Manual, 2nd ed., Vol. 1-3 (1989). The term “mutated” as used herein regarding genes means that at least one nucleotide in the nucleic acid sequence of that gene or a regulatory sequence thereof, has been replaced with a different nucleotide, or has been deleted from the sequence via mutagenesis, resulting in the transcription of a protein sequence with a qualitatively of quantitatively altered function or the knock-out of that gene. In the context of this invention an “altered gene” has the same meaning as a mutated gene.

The term “gene”, as used herein, refers to a nucleic acid sequence that can be transcribed into mRNAs that are then translated into protein. A gene encoding for a certain protein refers to the one or more nucleic acid sequence(s) encoding for such a protein.

The term “nucleic acid” as used herein, refers to a deoxyribonucleotide or ribonucleotide polymer, i.e. a polynucleotide, in either single or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including among other things, simple and complex cells.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. The terms “polypeptide”, “peptide” and “protein” are also inclusive of modifications including, but not limited to, glycosylation, lipid attachment, sulphation, gamma-carboxylation of glutamic acid residues, hydroxylation and ADP-ribosylation.

When an enzyme is mentioned with reference to an enzyme class (EC), the enzyme class is a class wherein the enzyme is classified or may be classified, on the basis of the Enzyme Nomenclature provided by the Nomenclature Committee of the International Union of Biochemistry and Molecular Biology (NC-IUBMB), which nomenclature may be found at http://www.chem.qmul.ac.uk/iubmb/enzyme/. Other suitable enzymes that have not (yet) been classified in a specified class but may be classified as such, are meant to be included.

If referred herein to a protein or a nucleic acid sequence, such as a gene, by reference to a accession number, this number in particular is used to refer to a protein or nucleic acid sequence (gene) having a sequence as can be found via www.ncbi.nlm.nih.gov/, (as available on 1 Nov. 2019) unless specified otherwise.

Every nucleic acid sequence herein that encodes a polypeptide also includes any conservatively modified variants thereof. This includes that, by reference to the genetic code, it describes every possible silent variation of the nucleic acid. The term “conservatively modified variants” applies to both amino acid and nucleic acid sequences. With respect to particular nucleic acid sequences, conservatively modified variants refers to those nucleic acids which encode identical or conservatively modified variants of the amino acid sequences due to the degeneracy of the genetic code. The term “degeneracy of the genetic code” refers to the fact that a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation.

Any exogenous gene coding for an enzyme herein comprises a nucleotide sequence coding for an amino acid sequence with at least 50, 60, 65, 70, 75, 80, 85, 86, 87, 88, 89, 90, 91, 92, 93, 94, 95, 96, 97, 98, 99 or 100% amino acid sequence identity with any of SEQ ID's NO: X, wherein SEQ ID NO:X is any of the protein sequences in the sequence listing of this application. The exogenous gene coding for an enzyme may also comprises a nucleotide sequence coding for an amino acid sequence having one or several substitutions, insertions and/or deletions as compared to the amino acid sequence of any of SEQ ID NO: X. Preferably the amino acid sequence has no more than 300, 250, 200, 150, 100, 75, 50, 40, 30, 20, 10, 9, 8, 7, 6, 5, 4, 3, 2, or 1 amino acid substitutions, insertions and/or deletions as compared to SEQ ID's NO: X.

Any exogenous gene coding for an enzyme herein comprises a nucleotide sequence with at least 40, 50, 60, 65, 70, 75, 80, 85, 86, 87, 88, 89, 90, 95, 96, 97, 98, 99% or 100% nucleotide (DNA) sequence identity with any of SEQ ID's NO: Y, wherein SEQ ID NO: Y is any of the nucleotide (DNA) sequences in the sequence listing of this application.

The term “functional homologue” (or in short “homologue”) of a polypeptide having a specific sequence (e.g. “SEQ ID NO: X”), as used herein, refers to a polypeptide comprising said specific sequence with the proviso that one or more amino acids are substituted, deleted, added, and/or inserted, and which polypeptide has (qualitatively) the same enzymatic functionality for substrate conversion. This functionality may be tested by use of an assay system comprising a recombinant cell comprising an expression vector for the expression of the homologue in yeast, said expression vector comprising a heterologous nucleic acid sequence operably linked to a promoter functional in the yeast and said heterologous nucleic acid sequence encoding the homologous polypeptide of which enzymatic activity for converting acetyl-Coenzyme A to acetaldehyde in the cell is to be tested, and assessing whether said conversion occurs in said cells. With respect to nucleic acid sequences, the term functional homologue is meant to include nucleic acid sequences which differ from another nucleic acid sequence due to the degeneracy of the genetic code and encode the same polypeptide sequence.

Sequence identity is herein defined as a relationship between two or more amino acid (polypeptide or protein) sequences or two or more nucleic acid (polynucleotide) sequences, as determined by comparing the sequences. Usually, sequence identities or similarities are compared over the whole length of the sequences compared. In the art, “identity” also means the degree of sequence relatedness between amino acid or nucleic acid sequences, as the case may be, as determined by the match between strings of such sequences.

Amino acid or nucleotide sequences are said to be homologous when exhibiting a certain level of similarity. Two sequences being homologous indicate a common evolutionary origin. Whether two homologous sequences are closely related or more distantly related is indicated by “percent identity” or “percent similarity”, which is high or low respectively. Although disputed, to indicate “percent identity” or “percent similarity”, “level of homology” or “percent homology” are frequently used interchangeably. A comparison of sequences and determination of percent identity between two sequences can be accomplished using a mathematical algorithm. The skilled person will be aware of the fact that several different computer programs are available to align two sequences and determine the homology between two sequences (Kruskal, J. B. (1983) An overview of sequence comparison In D. Sankoff and J. B. Kruskal, (ed.), Time warps, string edits and macromolecules: the theory and practice of sequence comparison, pp. 1-44 Addison Wesley). The percent identity between two amino acid sequences can be determined using the Needleman and Wunsch algorithm for the alignment of two sequences. (Needleman, S. B. and Wunsch, C. D. (1970) J. Mol. Biol. 48, 443-453). The algorithm aligns amino acid sequences as well as nucleotide sequences. The Needleman-Wunsch algorithm has been implemented in the computer program NEEDLE. For the purpose of this invention the NEEDLE program from the EMBOSS package was used (version 2.8.0 or higher, EMBOSS: The European Molecular Biology Open Software Suite (2000) Rice, P. Longden, I. and Bleasby, A. Trends in Genetics 16, (6) pp276-277, http://emboss.bioinformatics.nl/). For protein sequences, EBLOSUM62 is used for the substitution matrix. For nucleotide sequences, EDNAFULL is used. Other matrices can be specified. The optional parameters used for alignment of amino acid sequences are a gap-open penalty of 10 and a gap extension penalty of 0.5. The skilled person will appreciate that all these different parameters will yield slightly different results but that the overall percentage identity of two sequences is not significantly altered when using different algorithms.

The homology or identity is the percentage of identical matches between the two full sequences over the total aligned region including any gaps or extensions. The homology or identity between the two aligned sequences is calculated as follows: Number of corresponding positions in the alignment showing an identical amino acid in both sequences divided by the total length of the alignment including the gaps. The identity defined as herein can be obtained from NEEDLE and is labelled in the output of the program as “IDENTITY”.

The homology or identity between the two aligned sequences is calculated as follows: Number of corresponding positions in the alignment showing an identical amino acid in both sequences divided by the total length of the alignment after subtraction of the total number of gaps in the alignment. The identity defined as herein can be obtained from NEEDLE by using the NOBRIEF option and is labeled in the output of the program as “longest-identity”.

A variant of a nucleotide or amino acid sequence disclosed herein may also be defined as a nucleotide or amino acid sequence having one or several substitutions, insertions and/or deletions as compared to the nucleotide or amino acid sequence specifically disclosed herein (e.g. in de the sequence listing).

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. In an embodiment, conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. In an embodiment, conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gln; Ile to Leu or Val; Leu to Ile or Val; Lys to Arg; Gln or Glu; Met to Leu or Ile; Phe to Met, Leu or Tyr; Ser to Thr; Thr to Ser; Trp to Tyr; Tyr to Trp or Phe; and, Val to Ile or Leu.

Nucleotide sequences of the invention may also be defined by their capability to hybridise with parts of specific nucleotide sequences disclosed herein, respectively, under moderate, or preferably under stringent hybridisation conditions. Stringent hybridisation conditions are herein defined as conditions that allow a nucleic acid sequence of at least about 25, preferably about 50 nucleotides, 75 or 100 and most preferably of about 200 or more nucleotides, to hybridise at a temperature of about 65° C. in a solution comprising about 1 M salt, preferably 6×SSC or any other solution having a comparable ionic strength, and washing at 65° C. in a solution comprising about 0.1 M salt, or less, preferably 0.2×SSC or any other solution having a comparable ionic strength. Preferably, the hybridisation is performed overnight, i.e. at least for 10 hours and preferably washing is performed for at least one hour with at least two changes of the washing solution. These conditions will usually allow the specific hybridisation of sequences having about 90% or more sequence identity.

Moderate conditions are herein defined as conditions that allow a nucleic acid sequences of at least 50 nucleotides, preferably of about 200 or more nucleotides, to hybridise at a temperature of about 45° C. in a solution comprising about 1 M salt, preferably 6×SSC or any other solution having a comparable ionic strength, and washing at room temperature in a solution comprising about 1 M salt, preferably 6×SSC or any other solution having a comparable ionic strength. Preferably, the hybridisation is performed overnight, i.e. at least for 10 hours, and preferably washing is performed for at least one hour with at least two changes of the washing solution. These conditions will usually allow the specific hybridisation of sequences having up to 50% sequence identity. The person skilled in the art will be able to modify these hybridisation conditions in order to specifically identify sequences varying in identity between 50% and 90%.

“Expression” refers to the transcription of a gene into structural RNA (rRNA, tRNA) or messenger RNA (mRNA) with subsequent translation into a protein. “Overexpression” refers to expression of a gene by a recombinant cell in excess to its expression in a corresponding wild-type cell. Such overexpression can for example be arranged for by: increasing the frequency of transcription of one or more nucleic acid sequences, for example by operational linking of the nucleic acid sequence to a promoter functional within the recombinant cell; and/or by increasing the number of copies of a certain nucleic acid sequence.

In analogy with the above, one skilled in the art will understand that by a reduction, downregulation, inhibition and/or elimination of a certain activity of a certain protein, is meant that the activity in the recombinant cell of such protein has been reduced, downregulated, inhibited and/or eliminated as compared to a corresponding wild-type cell.

Nucleic acid sequences (i.e. polynucleotides) or proteins (i.e. polypeptides) may be homologous or heterologous to the genome of the host cell.

“Homologous” with respect to a host cell, means that the nucleic acid sequence does naturally occur in the genome of the host cell or that the protein is naturally produced by that cell. Homologous protein expression may e.g. be an overexpression or expression under control of a different promoter. In the present inventions the host cell is a yeast.

The term “heterologous”, with respect to the host cell, means that the polynucleotide does not naturally occur in the genome of the host cell or that the polypeptide is not naturally produced by that cell. Heterologous protein expression involves expression of a protein that is not naturally produced in the host cell. As used herein, “heterologous” may refer to a nucleic acid or protein is a nucleic acid or protein that originates from a foreign species, or, if from the same species, is substantially modified from its native form in composition and/or genomic locus by deliberate human intervention. For example, a promoter operably linked to a heterologous structural gene is from a species different from that from which the structural gene was derived, or, if from the same species, one or both are substantially modified from their original form. A heterologous protein may originate from a foreign species or, if from the same species, is substantially modified from its original form by deliberate human intervention.

The term “heterologous expression” refers to the expression of heterologous nucleic acids in a host cell. The expression of heterologous proteins in eukaryotic host cell systems such as yeast are well known to those of skill in the art. A polynucleotide comprising a nucleic acid sequence of a gene encoding an enzyme with a specific activity can be expressed in such a eukaryotic system. In some embodiments, transformed/transfected cells may be employed as expression systems for the expression of the enzymes. Expression of heterologous proteins in yeast is well known. Sherman, F., et al., Methods in Yeast Genetics, Cold Spring Harbor Laboratory (1982) is a well-recognized work describing the various methods available to express proteins in yeast. Two widely utilized yeasts are Saccharomyces cerevisiae and Pichia pastoris. Vectors, strains, and protocols for expression in Saccharomyces and Pichia are known in the art and available from commercial suppliers (e.g., Invitrogen). Suitable vectors usually have expression control sequences, such as promoters, including 3-phosphoglycerate kinase or alcohol oxidase, and an origin of replication, termination sequences and the like as desired.

As used herein “promoter” is a DNA sequence that directs the transcription of a (structural) gene. Typically, a promoter is located in the 5′-region of a gene, proximal to the transcriptional start site of a (structural) gene. Promoter sequences may be constitutive, inducible or repressible. In an embodiment there is no (external) inducer needed.

The term “vector” as used herein, includes reference to an autosomal expression vector and to an integration vector used for integration into the chromosome.

The term “expression vector” refers to a DNA molecule, linear or circular, that comprises a segment encoding a polypeptide of interest under the control of (i.e. operably linked to) additional nucleic acid segments that provide for its transcription. Such additional segments may include promoter and terminator sequences, and may optionally include one or more origins of replication, one or more selectable markers, an enhancer, a polyadenylation signal, and the like. Expression vectors are generally derived from plasmid or viral DNA, or may contain elements of both. In particular an expression vector comprises a nucleic acid sequence that comprises in the 5′ to 3′ direction and operably linked: (a) a yeast-recognized transcription and translation initiation region, (b) a coding sequence for a polypeptide of interest, and (c) a yeast-recognized transcription and translation termination region. “Plasmid” refers to autonomously replicating extrachromosomal DNA which is not integrated into a microorganism's genome and is usually circular in nature.

An “integration vector” refers to a DNA molecule, linear or circular, that can be incorporated in a microorganism's genome and provides for stable inheritance of a gene encoding a polypeptide of interest. The integration vector generally comprises one or more segments comprising a gene sequence encoding a polypeptide of interest under the control of (i.e. operably linked to) additional nucleic acid segments that provide for its transcription. Such additional segments may include promoter and terminator sequences, and one or more segments that drive the incorporation of the gene of interest into the genome of the target cell, usually by the process of homologous recombination. Typically, the integration vector will be one which can be transferred into the target cell, but which has a replicon which is nonfunctional in that organism. Integration of the segment comprising the gene of interest may be selected if an appropriate marker is included within that segment.

By “host cell” is herein understood a cell, such as a yeast cell, that is to be transformed with one or more nucleic acid sequences encoding for one or more heterologous proteins, to construe a transformed cell, also referred to as a recombinant cell. For example, the transformed cell may contain a vector and may support the replication and/or expression of the vector.

“Transformation” and “transforming”, as used herein, refers to the insertion of an exogenous polynucleotide (i.e. an exogenous nucleic acid sequence) into a host cell, irrespective of the method used for the insertion, for example, direct uptake, transduction, f-mating or electroporation. The exogenous polynucleotide may be maintained as a non-integrated vector, for example, a plasmid, or alternatively, may be integrated into the host cell genome.

By “disruption” is herein understood any disruption of activity, including, but not limited to, deletion, mutation and reduction of the affinity of the disrupted gene and expression of RNA complementary to such disrupted gene. It includes all nucleic acid modifications such as nucleotide deletions or substitutions, gene knock-outs, and other actions which affect the translation or transcription of the corresponding polypeptide and/or which affect the enzymatic (specific) activity, its substrate specificity, and/or or stability. It also includes modifications that may be targeted on the coding sequence or on the promotor of the gene. A gene disruptant is a cell that has one or more disruptions of the respective gene native to the yeast. Native to yeast herein is understood as that the gene is present in the yeast cell before the disruption.

The term “encoding” has the same meaning as “coding for”. Thus, by way of example, “one or more heterologous genes encoding a glycerol dehydrogenase” has the same meaning as “one or more heterologous genes coding for a glycerol dehydrogenase”. As far as genes encoding an enzyme are concerned, the phrase “one or more heterologous genes encoding a X”, wherein X denotes an enzyme, has the same meaning as “one or more heterologous genes encoding an enzyme having X activity”. Thus, by way of example, “one or more heterologous genes encoding a glycerol dehydrogenase” has the same meaning as “one or more heterologous genes encoding an enzyme having glycerol dehydrogenase activity”.

The abbreviation “NADH” refers to reduced, hydrogenated form of nicotinamide adenine dinucleotide. The abbreviation “NAD+” refers to the oxidized form of nicotinamide adenine dinucleotide. Nicotinamide adenine dinucleotide may act as a so-called cofactor, assisting in biochemical reactions and/or transformations in a cell.

By a NADH dependent enzyme is herein understood an enzyme that is exclusively depended on NADH as a co-factor or that is predominantly dependent on NADH as a cofactor. By an “exclusive NADH dependent” enzyme is herein understood an enzyme that has an absolute requirement for NADH over NADPH. That is, it is only active when NADH is applied as cofactor.

By a “predominantly NADH-dependent” enzyme is herein understood an enzyme that has a higher specificity and/or a higher catalytic efficiency for NADH as a cofactor than for NADPH as a cofactor.

The enzyme's specificity characteristics can be described by the formula:

1<KₘNADP⁺/KₘNAD⁺<∞(infinity)

wherein Kₘ is the so-called Michaelis constant.

For a predominantly NADH-dependent enzyme, preferably KₘNADP⁺/KₘNAD⁺ is between 1 and 1000, between 1 and 500, between 1 and 200, between 1 and 100, between 1 and 50, between 1 and 10, between 5 and 100, between 5 and 50, between 5 and 20 or between 5 and 10. The Kₘ's for the enzymes herein can be determined as enzyme specific, for NAD⁺ and NADP⁺ respectively, using know analysis techniques, calculations and protocols. These are described for instance in Lodish et al., Molecular Cell Biology 6^{th} Edition, Ed. Freeman, pages 80 and 81, e.g. FIGS. 3-22.

For an predominantly NADH-dependent enzyme, preferably the ratio of the catalytic efficiency for NADPH/NADP+ as a cofactor (k_{cat}/Kₘ)^{NADP+} to NADH/NAD+ as cofactor (k_{cat}/Kₘ)^{NAD+,} i.e. the catalytic efficiency ratio (k_{cat}/Kₘ)^{NADP+}L(k_{cat}/kₘ)^{NAD+}, is more than 1:1, more preferably equal to or more than 2:1, still more preferably equal to or more than 5:1, even more preferably equal to or more than 10:1, yet even more preferably equal to or more than 20:1, even still more preferably equal to or more than 100:1, and most preferably equal to or more than 1000:1.

There is no upper limit, but for practical reasons the predominantly NADH-dependent enzyme may have a catalytic efficiency ratio (k_{cat}/Kₘ)^{NADP+}:(k_{cat}/Kₘ)^{NAD+} of equal to or less than 1.000.000.000:1 (i.e. 1.10⁹:1).

By a “glucose-tolerant gene” or a “glucose-tolerant nucleic acid sequence” is herein understood a nucleic acid sequence encoding for the synthesis of an enzyme, which nucleic acid sequence does not suffer from glucose inactivation and/or glucose repression or where glucose inactivation or glucose repression is reduced. By a reduction is herein preferably understood a reduction as compared to a corresponding wild-type cell.

The Carbon Source Composition

By a carbon source composition is herein understood a composition containing one or more carbon sources. The carbon source composition according to the invention comprises at least glucose and arabinose. Optionally the carbon source composition may comprise further sugars such as xylose, galactose and/or other sugars. Preferably the carbon source composition further comprises glycerol and/or acetic acid and/or a salt thereof. A salt of acetic acid is herein also referred to as acetate. These different carbon sources can differ in amount of carbon contained. For example, glucose, arabinose, xylose and acetic acid comprise about 40 wt % carbon, whilst glycerol comprises about 39 wt % carbon. Product ethanol comprises about 52 wt % carbon.

Preferably the carbon source composition is derived from a cellulose and/or hemicellulose comprising material, such as for example a lignocellulosic material. Such a cellulose and/or hemicellulose comprising material may be hydrolysed into a hydrolysate, also referred to as a cellulosic and/or hemicellulosic hydrolysate. Such a hydrolysate can contain one or more monomeric sugars, such as the above mentioned glucose, arabinose, xylose, galactose and/or mannose. In addition, such a hydrolysate may comprise carbon sources other than sugars, such as for example glycerol and/or acetic acid. Preferably the carbon source composition comprises and/or is derived from a hydrolysate, preferably a cellulosic, hemicellulosic and/or lignocellulosic hydrolysate. The hydrolysate is preferably derived from biomass, more preferably from plant biomass.

Lignocellulosic materials can include cellulose, hemicellulose and/or lignin. Monomeric sugars can be derived from the cellulosic and/or hemicellulosic parts of a lignocellulosic material. Suitable lignocellulosic materials may be found in the following list: orchard primings, chaparral, mill waste, urban wood waste, municipal waste, logging waste, forest thinnings, short-rotation woody crops, industrial waste, wheat straw, oat straw, rice straw, barley straw, rye straw, flax straw, soy hulls, rice hulls, rice straw, corn gluten feed, oat hulls, sugar cane, corn stover, corn stalks, corn cobs, corn fiber, corn husks, switch grass, miscanthus, sweet sorghum, canola stems, soybean stems, prairie grass, gamagrass, foxtail; sugar beet pulp, citrus fruit pulp, seed hulls, cellulosic animal wastes, lawn clippings, cotton, seaweed, trees, softwood, hardwood, poplar, pine, shrubs, grasses, wheat, wheat straw, sugar cane bagasse, corn, corn husks, corn hobs, corn kernel, fiber from kernels, products and by-products from wet or dry milling of grains, municipal solid waste, waste paper, yard waste, herbaceous material, agricultural residues, forestry residues, municipal solid waste, waste paper, pulp, paper mill residues, branches, bushes, canes, corn, corn husks, an energy crop, forest, a fruit, a flower, a grain, a grass, a herbaceous crop, a leaf, bark, a needle, a log, a root, a sapling, a shrub, switch grass, a tree, a vegetable, fruit peel, a vine, sugar beet pulp, wheat midlings, oat hulls, hard or soft wood, organic waste material generated from an agricultural process, forestry wood waste, or a combination of any two or more thereof. Preferred examples of materials including hemicellulose and/or cellulose include corn cobs, corn fibre, rice hulls, melon shells, sugar beet pulp, wheat straw, sugar cane bagasse, wood, grass and olive pressings.

An overview of some suitable sugar compositions derived from cellulose and/or hemicellulose comprising material and the sugar composition of their hydrolysates is given in Table 1. In addition to sugars, such hydrolysates may comprise glycerol and/or acetic acid.

TABLE 1
Overview of sugar compositions from lignocellulosic materials.

| | | | | | | | | %. |
|---|---|---|---|---|---|---|---|---|
| Lignocellulosic material | Gal | Xyl | Ara | Man | Glu | Rham | Sum | Gal. |
| Corn cob a | 10 | 286 | 36 | | 227 | 11 | 570 | 1.7 |
| Corn cob b | 131 | 228 | 160 | | 144 | | 663 | 19.8 |
| Rice hulls a | 9 | 122 | 24 | 18 | 234 | 10 | 417 | 2.2 |
| Rice hulls b | 8 | 120 | 28 | | 209 | 12 | 378 | 2.2 |
| Melon Shells | 6 | 120 | 11 | | 208 | 16 | 361 | 1.7 |
| Sugar beet pulp | 51 | 17 | 209 | 11 | 211 | 24 | 523 | 9.8 |
| Wheat straw Idaho | 15 | 249 | 36 | | 396 | | 696 | 2.2 |
| Corn fiber | 36 | 176 | 113 | | 372 | | 697 | 5.2 |
| Cane Bagasse | 14 | 180 | 24 | 5 | 391 | | 614 | 2.3 |
| Corn stover | 19 | 209 | 29 | | 370 | | 626 | |
| Athel (wood) | 5 | 118 | 7 | 3 | 493 | | 625 | 0.7 |
| Eucalyptus (wood) | 22 | 105 | 8 | 3 | 445 | | 583 | 3.8 |
| CWR (grass) | 8 | 165 | 33 | | 340 | | 546 | 1.4 |
| JTW (grass) | 7 | 169 | 28 | | 311 | | 515 | 1.3 |
| MSW | 4 | 24 | 5 | 20 | 440 | | 493 | 0.9 |
| Reed Canary Grass Veg | 16 | 117 | 30 | 6 | 209 | 1 | 379 | 4.2 |
| Reed Canary Grass Seed | 13 | 163 | 28 | 6 | 265 | 1 | 476 | 2.7 |
| Olive pressing residu | 15 | 111 | 24 | 8 | 329 | | 487 | 3.1 |

Gal = galactose, Xyl = xylose, Ara = arabinose, Man = mannose, Glu = glutamate, Rham = rhamnose.
The percentage galactose (% Gal).

Recombinant Yeast

The “recombinant yeast” may herein also be referred to as the “recombinant yeast cell” or simply as “yeast cell” or “host cell”. Yeasts are known to be eukaryotic microorganisms and include all species of the subdivision Eumycotina (Yeasts: characteristics and identification, J. A. Barnett, R. W. Payne, D. Yarrow, 2000, 3rd ed., Cambridge University Press, Cambridge UK; and, The yeasts, a taxonomic study, C. P. Kurtzman and J. W. Fell (eds) 1998, 4th ed., Elsevier Science Publ. B.V., Amsterdam, The Netherlands) which predominantly grow in unicellular form. Yeasts may either grow by budding of a unicellular thallus or may grow by fission of the organism. Preferred yeasts cells for use in the present inventions belong to the genera Saccharomyces, Kluyveromyces, Candida, Pichia, Zygosaccharomyces, Brettanomyces, Issatchenkia, Schizosaccharomyces, Hansenula, Kloeckera, Schwanniomyces, and Yarrowia. That is, preferably the recombinant yeast is a yeast from one of the genera Saccharomyces, Kluyveromyces, Candida, Pichia, Zygosaccharomyces, Brettanomyces, Issatchenkia, Schizosaccharomyces, Hansenula, Kloeckera, Schwanniomyces, and Yarrowia.

Preferably the yeast cell is capable of anaerobic fermentation, more preferably anaerobic alcoholic fermentation.

Due to the many attractive features of Saccharomyces species for industrial processes, including a high acid-, ethanol- and osmo-tolerance, capability of anaerobic growth, and of course its high alcoholic fermentative capacity, more preferably the recombinant yeast is derived from a yeast belonging to the genus Saccharomyces. Still more preferably the recombinant yeast is derived from a yeast chosen from the group consisting of S. cerevisiae, S. exiguus, S. bayanus, K. lactis, K. marxianus and Schizosaccharomyces pombe. Most preferably the recombinant yeast is derived from the yeast Saccharomyces cerevisiae.

The yeast cell may suitably act as a host cell, that may be transformed with one or more nucleic acid constructs comprising the genes encoding for any heterologous proteins to construe the recombinant yeast. The genes and/or nucleic acid sequences encoding for any such heterologous proteins are described below one by one. The recombinant yeast may comprise any suitable combination of the activities, genes and/or nucleic acid sequences as described hereinbelow.

Arabinose Isomerase, Ribulokinase and Ribulose Phosphate Epimerase

The recombinant yeast in the invention suitably comprises arabinose isomerase (araA) activity, ribulokinase (araB) activity and ribulose phosphate epimerase (araD) activity.

Preferably the recombinant yeast comprises: one or more nucleic acid sequences encoding for a protein having arabinose isomerase (araA) activity; one or more nucleic acid sequences encoding for a protein having ribulokinase (araB) activity; and one or more nucleic acid sequences encoding for a protein having ribulose phosphate epimerase (araD) activity. More preferably the recombinant yeast comprises a bacterial gene encoding a heterologous protein with arabinose isomerase activity; a bacterial gene encoding a heterologous protein with ribulokinase activity; and a bacterial gene encoding a heterologous protein with ribulose phosphate epimerase activity, suitably allowing the recombinant yeast to functionally express such proteins.

The recombinant yeast may therefore be capable of converting L-arabinose into L-ribulose and/or xylulose 5-phosphate and/or into a desired fermentation product such as ethanol.

Recombinant yeasts, for example derived from S. cerevisiae yeast strains, able to produce ethanol from L-arabinose may be produced by modifying a cell introducing the araA (L-arabinose isomerase), araB (L-ribulokinase) and araD (L-ribulose-5-P4-epimerase) genes from a suitable source. Such genes may be introduced to make the yeast capable of using arabinose. Methods for introduction of such genes are known in the art and described in WO2003/095627, WO2011/003893 and WO2011/131667, incorporated herein by reference. Preferably araA, araB and araD genes from Lactobacillus plantanum may be used, or a functional homologue thereof having a nucleic acid sequence with at least 60, 65, 70, 75, 80, 85, 90, 95, 98, 99% nucleic acid sequence identity therewith, as described in WO2008/041840 and herein incorporated by reference. The araA gene from Bacillus subtilis, or a functional homologue thereof having a nucleic acid sequence with at least 60, 65, 70, 75, 80, 85, 90, 95, 98, 99% nucleic acid sequence identity therewith, and the araB and araD genes from Escherichia coli may also be used and are disclosed in EP1499708, incorporated herein by reference. It is further possible for the araA, araB and araD genes to be derived from at least one of the genus Clavibacter, Arthrobacter and/or Gramella, in particular one of Clavibacter michiganensis, Arthrobacter aurescens, and/or Gramella forsetii, as disclosed in WO 2009011591.

The recombinant yeast may comprise one or more copies of the araA, araB and/or araD genes. Preferably the recombinant yeast comprises in the range from 2 to 15, more preferably in the range from 3 to 10 copies of the araA, araB and/or araD genes.

Preferably the araA, araB and/or araD genes are incorporated in the genome of the recombinant yeast.

The recombinant yeast may further comprise a nucleic acid sequence encoding a heterologous protein having arabinose transporter (araT) activity.

The nucleic acid sequence may for example comprise an araT gene derived from an organism selected from the group consisting of Ambrosiozyma monospora (LAT2), Candida arabinofermentans, Ambrosiozyma monospora (LAT1), Kluveromyces marxianus (LAT1), Pichia guillermondii (LAT1), Pichia guillermondii (LAT2), Pichia stipites, Ambrosiozyma monospora (LAT2), Debaryomyces hensenii, Apergillus flavus, Aspergillus terreus, Neosartorya fischeri, Aspergillus niger. Penicillium marneffei, Coccidioides posadasii, Gibberella zeae, Magnaporthe oryzae, Schizophyllum commune, Pichia stipites, Saccharomyces HXT2, Aspergillus clavatus (ACLA_032060), Sclerotinia sclerotiorum (SS1G_01302), Arthroderma benhamiae (ARB_03323), Trichophyton equinum (TEQG_03356), Trichophyton tonsurans (G_04876), Coccidioides immitis (CIMG 0.09387), Coccidioides posadasii (CPSG_03942), Coccidioides posadasii (CPC735_017640), Botryotinia fuckeliana (BC 1G_08389), Pyrenophora tritici-repentis (PTRG_10527), Ustilago maydis (UM03895.1), Clavispora lusitaniae (CLUG_02297), Pichia guillermondii (LAT1), Pichia guillermondii (LAT2), Debaryomyces hansenii (DEHA2E01166g), Pichia stipites, Candida albicans, Debaryomyces hansenii (DEHA2B16082g), Kluveromyces marxianus (LAT1), Kluyveromyces lactis (KLLA-ORF 10059), Lachancea thermotolerans (KLTH0H13728g), Vanderwaltozyma polyspora (Kpol_281p3), Zygosaccharomyces rouxii (ZYRO0E03916g), Pichia pastoris (0.1833), Candida arabinofermentans (0.1378), Ambrosiozyma monospora (LAT1), Aspergillus clavatus (ACLA_044740), Neosartorya fischeri (NFIA_094320), Aspergillus flavus (AFLA_116400), Aspergillus terreus (ATEG_08609), Aspergillus niger (ANI_1_1064034), Telaromyces stipitatus (TSTA 124770), Penicillium chrysogenum (Pc20g01790), Penicillium chrysogenum (Pc20g01790)#2, Gibberella zeae (FG10921.1), Nectria hematococco, and Glomerella graminicola (GLRG_10740), or a functional homologue of any of these having a nucleic acid sequence with at least 60, 65, 70, 75, 80, 85, 90, 95, 98, 99% nucleic acid sequence identity therewith.

Most preferably the recombinant yeast comprises:
a gene encoding for a heterologous protein comprising an amino acid sequence represented by SEQ ID NO: 10 herein or a functional homologue of SEQ ID NO:10 herein having a sequence identity of at least 60%, at least 65%, at least 70%, at least 75%, at least 80%, at least 85%, at least 90%, at least 95%, at least 98% or at least 99% with SEQ ID No:10 herein; and/or
a gene encoding for a protein comprising an amino acid sequence represented by SEQ ID NO: 11 herein or a functional homologue of SEQ ID NO:11 herein having a sequence identity of at least 60%, at least 65%, at least 70%, at least 75%, at least 80%, at least 85%, at least 90%, at least 95%, at least 98% or at least 99% with SEQ ID No:11 herein; and/or
a gene encoding for a protein comprising an amino acid sequence represented by SEQ ID NO: 12 herein or a functional homologue of SEQ ID NO:12 herein having a sequence identity of at least 60%, at least 65%, at least 70%, at least 75%, at least 80%, at least 85%, at least 90%, at least 95%, at least 98% or at least 99% with SEQ ID No:12 herein.

It is also possible for the recombinant yeast to comprise a so-called GAL2 transporter. Preferences for such GAL2 transporter are as described in WO2014195376A3, incorporated herein by reference.

PPP-Genes

The recombinant yeast in the invention may further comprise one or more genetic modifications that increases the flux of the pentose phosphate pathway. The genes encoding for this pentose phosphate pathway are herein also referred to as the “PPP” genes.

In a preferred host cell, the genetic modification comprises overexpression of at least one enzyme of the (non-oxidative part) pentose phosphate pathway. Preferably the enzyme is selected from the group consisting of the enzymes encoding for ribulose-5-phosphate isomerase, ribulose-5-phosphate epimerase, transketolase and transaldolase. Various combinations of enzymes of the (non-oxidative part) pentose phosphate pathway may be overexpressed. E.g. the enzymes that are overexpressed may be at least the enzymes ribulose-5-phosphate isomerase and ribulose-5-phosphate epimerase; or at least the enzymes ribulose-5-phosphate isomerase and transketolase; or at least the enzymes ribulose-5-phosphate isomerase and transaldolase; or at least the enzymes ribulose-5-phosphate epimerase and transketolase; or at least the enzymes ribulose-5-phosphate epimerase and transaldolase; or at least the enzymes transketolase and transaldolase; or at least the enzymes ribulose-5-phosphate epimerase, transketolase and transaldolase; or at least the enzymes ribulose-5-phosphate isomerase, transketolase and transaldolase; or at least the enzymes ribulose-5-phosphate isomerase, ribulose-5-phosphate epimerase, and transaldolase; or at least the enzymes ribulose-5-phosphate isomerase, ribulose-5-phosphate epimerase, and transketolase.

Possibly each of the enzymes ribulose-5-phosphate isomerase, ribulose-5-phosphate epimerase, transketolase and transaldolase are overexpressed in the host cell. More preferred is a host cell in which the genetic modification comprises at least overexpression of both the enzymes transketolase and transaldolase.

The enzyme “ribulose 5-phosphate epimerase” (EC 5.1.3.1) is herein defined as an enzyme that catalyses the epimerisation of D-xylulose 5-phosphate into D-ribulose 5-phosphate and vice versa. The enzyme is also known as phosphoribulose epimerase; erythrose-4-phosphate isomerase; phosphoketopentose 3-epimerase; xylulose phosphate 3-epimerase; phosphoketopentose epimerase; ribulose 5-phosphate 3-epimerase; D-ribulose phosphate-3-epimerase; D-ribulose 5-phosphate epimerase; D-ribulose-5-P 3-epimerase; D-xylulose-5-phosphate 3-epimerase; pentose-5-phosphate 3-epimerase; or D-ribulose-5-phosphate 3-epimerase. A ribulose 5-phosphate epimerase may be further defined by its amino acid sequence. Likewise a ribulose 5-phosphate epimerase may be defined by a nucleotide sequence encoding the enzyme as well as by a nucleotide sequence hybridising to a reference nucleotide sequence encoding a ribulose 5-phosphate epimerase. The nucleotide sequence encoding for ribulose 5-phosphate epimerase is herein designated RPE1.

The enzyme “ribulose 5-phosphate isomerase” (EC 5.3.1.6) is herein defined as an enzyme that catalyses direct isomerisation of D-ribose 5-phosphate into D-ribulose 5-phosphate and vice versa. The enzyme is also known as phosphopentosisomerase; phosphoriboisomerase; ribose phosphate isomerase; 5-phosphoribose isomerase; D-ribose 5-phosphate isomerase; D-ribose-5-phosphate ketol-isomerase; or D-ribose-5-phosphate aldose-ketose-isomerase. A ribulose 5-phosphate isomerase may be further defined by its amino acid sequence. Likewise a ribulose 5-phosphate isomerase may be defined by a nucleotide sequence encoding the enzyme as well as by a nucleotide sequence hybridising to a reference nucleotide sequence encoding a ribulose 5-phosphate isomerase. The nucleotide sequence encoding for ribulose 5-phosphate isomerase is herein designated RKI1.

The enzyme “transketolase” (EC 2.2.1.1) is herein defined as an enzyme that catalyses the reaction: D-ribose 5-phosphate+D-xylulose 5-phosphate<->sedoheptulose 7-phosphate+D-glyceraldehyde 3-phosphate and vice versa. The enzyme is also known as glycolaldehydetransferase or sedoheptulose-7-phosphate:D-glyceraldehyde-3-phosphate glycolaldehydetransferase. A transketolase may be further defined by its amino acid. Likewise a transketolase may be defined by a nucleotide sequence encoding the enzyme as well as by a nucleotide sequence hybridising to a reference nucleotide sequence encoding a transketolase. The nucleotide sequence encoding for transketolase is herein designated TKL1.

The enzyme “transaldolase” (EC 2.2.1.2) is herein defined as an enzyme that catalyses the reaction: sedoheptulose 7-phosphate+D-glyceraldehyde 3-phosphate<->D-erythrose 4-phosphate+D-fructose 6-phosphate and vice versa. The enzyme is also known as dihydroxyacetonetransferase; dihydroxyacetone synthase; formaldehyde transketolase; or sedoheptulose-7-phosphate:D-glyceraldehyde-3-phosphate glyceronetransferase. A transaldolase may be further defined by its amino acid sequence. Likewise a transaldolase may be defined by a nucleotide sequence encoding the enzyme as well as by a nucleotide sequence hybridising to a reference nucleotide sequence encoding a transaldolase. The nucleotide sequence encoding for transketolase from is herein designated TAL1.

Xylose Isomerase or Xylose Reductase and Xylitol Dehydrogenase Genes

In addition to the glucose and arabinose, the carbon source composition may further comprise xylose. Xylose is a carbon source similar to glucose and arabinose. If the carbon source composition further comprises xylose, the recombinant yeast preferably comprises, one, two or more copies of a heterologous gene encoding for one or more protein(s) having xylose isomerase activity; and/or one, two or more copies of a heterologous gene encoding for one or more protein(s) having xylose reductase activity; and/or one, two or more copies of a heterologous gene encoding for one or more protein(s) having xylitol dehydrogenase activity.

The presence of these two or more genetic elements confers on the recombinant yeast the ability to convert xylose by isomerisation or reduction.

A “xylose isomerase” (EC 5.3.1.5) is herein defined as an enzyme that catalyses the direct isomerisation of D-xylose into D-xylulose and/or vice versa. The enzyme is also known as a D-xylose ketoisomerase. A xylose isomerase herein may also be capable of catalysing the conversion between D-glucose and D-fructose (and accordingly may therefore be referred to as a glucose isomerase). A xylose isomerase herein may require a bivalent cation, such as magnesium, manganese or cobalt as a cofactor. Accordingly, a recombinant yeast comprising such xylose isomerase can be capable of isomerising xylose to xylulose. The ability of isomerising xylose to xylulose is conferred on the host cell by transformation of the host cell with a nucleic acid construct comprising a nucleotide sequence encoding a defined xylose isomerase. A recombinant yeast can isomerize xylose into xylulose by the direct isomerisation of xylose to xylulose. The Xylose isomerase gene may have various origins, such as for example Pyromyces sp. as disclosed in WO2006/009434 and herein incorporated by reference. Other suitable origins are Bacteroides, in particular Bacteroides uniformis as described in PCT/EP2009/52623 and herein incorporated by reference, Bacillus, in particular Bacillus stearothermophilus as described in PCT/EP2009/052625 and herein incorporated by reference.

It is also possible for two or more copies of one or more xylose reductase and/or xylitol dehydrogenase genes to be introduced into the genome of the recombinant yeast. In this case the conversion of xylose can be conducted in a two-step conversion of xylose into xylulose via a xylitol intermediate as catalysed by xylose reductase and xylitol dehydrogenase, respectively. For example, xylose reductase (XR), xylitol dehydrogenase (XDH), and xylokinase (XK) may be overexpressed, and optionally one or more of genes encoding NADPH producing enzymes are up-regulated and/or one or more of the genes encoding NADH consuming enzymes are up-regulated, as disclosed in WO 2004085627 incorporated herein by reference.

XKS1 Gene

The recombinant yeast in the invention may further comprise one or more genetic modifications that increase the specific xylulose kinase activity. Preferably the genetic modification or modifications causes overexpression of a xylulose kinase, e.g. by overexpression of a nucleotide sequence encoding a xylulose kinase. The gene encoding the xylulose kinase may be endogenous to the host cell or may be a xylulose kinase that is heterologous to the host cell. A nucleotide sequence used for overexpression of xylulose kinase in the host cell of the invention is a nucleotide sequence encoding a polypeptide with xylulose kinase activity.

The enzyme “xylulose kinase” (EC 2.7.1.17) is herein defined as an enzyme that catalyses the reaction ATP+D-xylulose=ADP+D-xylulose 5-phosphate. The enzyme is also known as a phosphorylating xylulokinase, D-xylulokinase or ATP:D-xylulose 5-phosphotransferase. A xylulose kinase of the invention may be further defined by its amino acid sequence. Likewise a xylulose kinase may be defined by a nucleotide sequence encoding the enzyme as well as by a nucleotide sequence hybridising to a reference nucleotide sequence encoding a xylulose kinase.

A genetic modification or modifications that increase(s) the specific xylulose kinase activity may be combined with any of the modifications increasing the flux of the pentose phosphate pathway as described above. This is not, however, essential.

Thus, a recombinant yeast in the invention may comprise only a genetic modification or modifications that increase the specific xylulose kinase activity. The various means available in the art for achieving and analysing overexpression of a xylulose kinase in the host cells of the invention are the same as described above for enzymes of the pentose phosphate pathway. Preferably in the host cells of the invention, a xylulose kinase to be overexpressed is overexpressed by at least a factor of about 1.1, about 1.2, about 1.5, about 2, about 5, about 10 or about 20 as compared to a strain which is genetically identical except for the genetic modification(s) causing the overexpression. It is to be understood that these levels of overexpression may apply to the steady state level of the enzyme's activity, the steady state level of the enzyme's protein as well as to the steady state level of the transcript coding for the enzyme.

Aldose Reductase (GRE3) Gene Deletion

If XI is used as gene to convert xylose, it may be advantageous to reduce aldose reductase activity. The recombinant yeast may in such a case comprise one or more genetic modifications that reduce unspecific aldose reductase activity in the host cell. Preferably, unspecific aldose reductase activity is reduced in the host cell by one or more genetic modifications that reduce the expression of or inactivates a gene encoding an unspecific aldose reductase. Preferably, the genetic modification(s) reduce or inactivate the expression of each endogenous copy of a gene encoding an unspecific aldose reductase in the host cell (herein called GRE3 deletion). Host cells may comprise multiple copies of genes encoding unspecific aldose reductases as a result of di-, poly- or aneu-ploidy, and/or the host cell may contain several different (iso)enzymes with aldose reductase activity that differ in amino acid sequence and that are each encoded by a different gene. Also in such instances preferably the expression of each gene that encodes an unspecific aldose reductase is reduced or inactivated. Preferably, the gene is inactivated by deletion of at least part of the gene or by disruption of the gene, whereby in this context the term gene also includes any non-coding sequence up- or down-stream of the coding sequence, the (partial) deletion or inactivation of which results in a reduction of expression of unspecific aldose reductase activity in the host cell.

Preferably, a nucleotide sequence encoding an aldose reductase whose activity is to be reduced in the recombinant yeast is a nucleotide sequence encoding a polypeptide with aldose reductase activity.

The enzyme “aldose reductase” (EC 1.1.1.21) is herein defined as any enzyme that is capable of reducing xylose or xylulose to xylitol. In the context of the present invention an aldose reductase may be any unspecific aldose reductase that is native (endogenous) to a host cell of the invention and that is capable of reducing xylose or xylulose to xylitol. Unspecific aldose reductases catalyse the reaction:

aldose+NAD(P)H+H⁺↔alditol+NAD(P)⁺

The enzyme has a wide specificity and is also known as aldose reductase; polyol dehydrogenase (NADP+); alditol:NADP oxidoreductase; alditol:NADP⁺ 1-oxidoreductase; NADPH-aldopentose reductase; or NADPH-aldose reductase.

A particular example of such an unspecific aldose reductase that is endogenous to S. cerevisiae and that is encoded by the GRE3 gene (Traff et al., 2001, Appl. Environ. Microbiol. 67: 5668-74). Thus, an aldose reductase of the invention may be further defined by its amino acid sequence. Likewise an aldose reductase may be defined by the nucleotide sequences encoding the enzyme as well as by a nucleotide sequence hybridising to a reference nucleotide sequence encoding an aldose reductase.

Glycerol-Uptake Proteins and Glycerol-Proton Symporter

The recombinant yeast suitably comprises glycerol uptake activity. By glycerol uptake activity is herein understood the activity of transporting glycerol from the medium into the yeast cell.

As explained in detail below, preferably the recombinant yeast comprises glycerol-proton symporter activity. More preferably the recombinant yeast comprises one or more nucleic acid sequences encoding for a protein having glycerol-proton symporter activity. Still more preferably the recombinant yeast comprises a glucose-tolerant gene encoding a heterologous protein with glycerol-proton symporter activity, suitably allowing the recombinant yeast to functionally express such a protein.

Nowadays many glycerol transporters (such as channels, facilitators and symporters) have been identified, characterized biochemically and the corresponding genes have been cloned (Neves, 2004, incorporated herein by reference).

As explained in WO2015/028583, in case of S. cerevisiae, four different genes have been implicated with glycerol transport (see Table 4 of WO2015/028583, incorporated herein by reference): FPS1, GUP1, GUP2 and STL1.

Fora number of reasons as explained in WO2015/028583, overexpression of one of these four S. cerevisiae membrane proteins is not expected to facilitate the uptake of glycerol across the plasma membrane under fermentation conditions. FPS1, GUP1 and GUP2 do not play a role in the uptake of glycerol. STL1 from S. cerevisiae encodes a glycerol-proton symporter, but is believed to be subject to glucose-repression at the transcription level and glucose-inactivation at the protein level.

In WO2015/028583, five alternative proteins were selected, heterologous to S. cerevisiae. These glycerol transporters, either being a facilitator, a channel, a uniporter or a symporter, were shown, upon overexpression in strains having anaerobic glycerol and acetic acid conversion pathways, to result in improved glycerol uptake activity in yeast cells.

Preferably the recombinant yeast in the present inventions comprises one or more nucleic acid sequence(s) and/or corresponding proteins as listed in Table 2 below, or a functional homologue of any of these having a nucleic acid sequence, respectively amino acid sequence, with at least 50, 55, 60, 65, 70, 75, 80, 85, 90, 95, 98, 99% nucleic acid sequence identity, respectively amino acid sequence, therewith. Specific examples of suitable protein(s) having glycerol uptake activity and their sequence identity with the protein first listed are summarized in Table 3(a) to 3(e).

TABLE 2
Preferred Glycerol-uptake proteins and genes encoding for such:

| Species | Gene Name | # AA | Protein Sequence |
|---|---|---|---|
| Schizosaccharomyces | SPAC977.17 | 598 | MSVPLRFSTPSSSPSASDNESVHDD |
| pombe | | | GPTTELDTFNTTDVPRRVNTTKARQ |
| SEQ ID NO: 1 | | | MRPKNTLKVAFSSPNLKGLDNTADS |
| | | | DSQPWLGGYLAGRLEDISGQSRRN |
| | | | YVDPYYEELNAGRRPNKPVWSLNG |
| | | | PLPHVLGNSVVEKISQKNQEARSRA |
| | | | NSRVNSRANSRANSSVSLAGMDGS |
| | | | PNWKRKMKSAVFGSRVKLNDEEAQ |
| | | | LPRNKSSVSIAEQAASRPKVSFSLQ |
| | | | SSRQPSIAEEQPQTQRKSSAITVEH |
| | | | AENAEPETPRNNVSFSRKPSIAEQD |
| | | | SSQDITMPPNEIIAEESLDSGSDTET |
| | | | LYLNYWCKIRHFFREGFAEFLGTLVL |
| | | | VVFGVGSNLQATVTNGAGGSFESLS |
| | | | FAWGFGCMLGVYIAGGISGGHVNPA |
| | | | VTISLAIFRKFPWYKVPIYIFFQIWGA |
| | | | FFGGALAYGYHWSSITEFEGGKDIR |
| | | | TPATGGCLYTNPKPYVTWRNAFFDE |
| | | | FIGTAVLVGCLFAILDDTNSPPTQGM |
| | | | TAFIVGLLIAAIGMALGYQTSFTLNPA |
| | | | RDLGPRMFAWWIGYGPHSFHLYHW |
| | | | WWTWGAWGGTIGGGIAGGLIYDLVI |
| | | | FTGPESPLNYPDNGFIDKKVHQITAK |
| | | | FEKEEEVENLEKTDSPIENN |
| Plasmodium falciparum | CAC88373 | 258 | MHMLFYKSYVREFIGEFLGTFVLMFL |
| SEQ ID NO: 2 | | | GEGATANFHTTGLSGDWYKLCLGW |
| | | | GLAVFFGILVSAKLSGAHLNLAVSIGL |
| | | | SSINKFDLKKIPVYFFAQLLGAFVGT |
| | | | STVYGLYHGFISNSKIPQFAWETSRN |
| | | | PSISLTGAFFNELILTGILLLVILVVVD |
| | | | ENICGKFHILKLSSVVGLIILCIGITFG |
| | | | GNTGFALNPSRDLGSRFLSLIAYGK |
| | | | DTFTKDNFYFWVPLVAPCVGSVVFC |
| | | | QFYDKVICPLVDLANNEKDGVDL |
| Danio rerio | AQP9 | 291 | MEYLENIRNLRGRCVLRRDIIREFLA |
| SEQ ID NO: 3 | NP_001171215 | | ELLGTFVLILFGCGSVAQTVLSREAK |
| | | | GQLLTIHFGFTLGVMLAVYMAGGVS |
| | | | GGHVNPAVSLAMVVLRKLPLKKFPV |
| | | | YVLAQFLGAFFGSCAVYCLYYDAFT |
| | | | EFANGELAVTGPNVTAGIFASYPRE |
| | | | GLSLLNGFIDQVIGAGALVLCILAVVD |
| | | | KKNIGAPKGMEPLLVGLSILAIGVSM |
| | | | ALNCGYPINPARDLGPRLFTAIAGW |
| | | | GLTVFSAGNGWWWVPVVGPMVGG |
| | | | VVGAAIYFLMIEMHHPENDKNLEDD |
| | | | NSLKDKYELNTVN |
| Xenopus tropicalis | NP_001087946 | 292 | MGRQKDFVNKCNQMLRLRNKLLRQ |
| SEQ ID NO: 4 | | | ALSECLGTLILVMFGCGSVAQVVLSK |
| | | | GSHGQFLTVNLAFGFAVMLGILISGQ |
| | | | VSGGHLNPAVTFALCILAREPWVKF |
| | | | PVYSIAQTLGAFLGAGIIYGLYYDAIW |
| | | | YFANDQLYVTGENGTAGIFTTFPSD |
| | | | HLTLMNGFFDQFIGTAALVVCVLAIV |
| | | | DPNNNPIPRGLEAFTVGFVVLVIGTS |
| | | | MGFNSGYAVNPARDFGPRLFTSLA |
| | | | GWGTEVFWAGNQWWWVPIVSPLL |
| | | | GAFAGVLVYQLMIGCHIEPVPQSTE |
| | | | QENIKLADVKPKDRI |
| Zygosaccharomyces | ZYRO0E01210p | 592 | MGKRTQGFMDYVFSRTSTAGLKGA |
| rouxii | | | RLRYTAAAVAVIGFALFGYDQGLMS |
| SEQ ID NO: 5 | | | GLITGDQFNKEFPPTKSNGDNDRYA |
| | | | SVIQGAVTACYEIGCFFGSLFVLFFG |
| | | | DAIGRKPLIIFGAIIVIIGTVISTAPFHH |
| | | | AWGLGQFVVGRVITGVGTGFNTSTI |
| | | | PVWQSEMTKPNIRGAMINLDGSVIA |
| | | | FGTMIAYWLDFGFSFINSSVQWRFP |
| | | | VSVQIIFALVLLFGIVRMPESPRWLM |
| | | | AKKRPAEARYVLACLNDLPENDDAIL |
| | | | AEMTSLHEAVNRSSNQKSQMKSLF |
| | | | SMGKQQNFSRALIASSTQFFQQFTG |
| | | | CNAAIYYSTVLFQTTVQLDRLLAMIL |
| | | | GGVFATVYTLSTLPSFYLVEKVGRR |
| | | | KMFFFGALGQGISFIITFACLVNPTKQ |
| | | | NAKGAAVGLYLFIICFGLAILELPWIY |
| | | | PPEIASMRVRAATNAMSTCTNWVTN |
| | | | FAVVMFTPVFIQTSQWGCYLFFAVM |
| | | | NFIYLPVIFFFYPETAGRSLEEIDIIFA |
| | | | KAHVDGTLPWMVAHRLPKLSMTEV |
| | | | EDYSQSLGLHDDENEKEEYDEKEAE |
| | | | ANAALFQVETSSKSPSSNRKDDDAP |
| | | | IEHNEVQESNDNSSNSSNVEAPIPV |
| | | | HHNDP |

TABLE 3 a)
SPAC977.17 from Schizosaccharomyces pombe and
proteins with a similar amino acid sequence identity.

| | Identity | Accession |
|---|---|---|
| Description | (%) | number |
| MIP water channel (predicted) | 100 | NP_592788.1 |
| [Schizosaccharomyces | | |
| pombe 972h-] >sp\|Q9P7T9.1\|YI7H | | |
| hypothetical protein TDEL_0C00200 | 54 | XP_003680120.1 |
| [Torulaspora delbrueckii] >emb\|CCE90909.1 | | |
| KLTH0B00440p [Lachancea thermotolerans] | 55 | XP_002551817.1 |
| >emb\|CAR21378.1 | | |
| YFL054C-like protein [Saccharomyces | 55 | EHN02649.1 |
| cerevisiae × Saccharomyces kudriavzevii | | |
| VIN7] | | |
| BN860_19284g1_1 [Zygosaccharomyces | 53 | CDF87997.1 |
| bailii CLIB 213] | | |
| aquaglyceroporin, putative [Aspergillus | 38 | EDP47128.1 |
| fumigatus A1163] | | |

TABLE 3 b)
CAC88373 from Plasmodium falciparum and proteins
with a similar amino acid sequence identity.

| | Identity | Accession |
|---|---|---|
| Description | (%) | number |
| aquaglyceroporin [Plasmodium | 100 | XP_001348009.1 |
| falciparum 3D7] >pdb\|3C02 | | |
| putative aquaglyceroporin [Plasmodium | 66 | ADD39010.1 |
| vivax] | | |
| glycerol uptake facilitator protein | 38 | WP_004881860.1 |
| [Pseudomonas viridiflava] | | |
| >gb\|EKN47076.1 | | |
| glycerol transporter [Pseudomonas | 38 | WP_007248745.1 |
| syringae group genomosp. 3] | | |
| >gb\|EGH58018.1 | | |
| glycerol uptake facilitator GIpF | 33 | WP_008907471.1 |
| [Caloramator australicus] | | |
| >emb\|CCC57748.1 | | |
| PREDICTED: aquaporin-10 | 33 | XP_004026862.1 |
| [Gorilla gorilla gorilla] | | |

TABLE 3 c)
AQP9 (NP_001171215) from Danio rerio and proteins
with a similar amino acid sequence identity.

| | Identity | Accession |
|---|---|---|
| Description | (%) | number |
| aquaporin-9 [Danio rerio] >gb\|ACB10576.1\| | 100 | NP_001171215.1 |
| aquaporin-9b [Danio rerio] | | |
| aquaglyceroporin [Osmerus mordax] | 73 | ABG24574.1 |
| PREDICTED: aquaporin-9 [Gorilla | 51 | XP_004056310.1 |
| gorilla gorilla] | | |
| aquaporin 3 (Gill blood group) | 52 | NP_001081876.1 |
| [Xenopus laevis] | | |
| >emb\|CAA10517.1 aquaporin-3 | | |
| [Xenopus laevis] | | |

TABLE 3 d)
NP_001087946 from Xenopus tropicalus and proteins
with a similar amino acid sequence identity.

| | Identity | Accession |
|---|---|---|
| Description | (%) | number |
| aquaporin 3 (Gill blood group) | 100 | NP_001087946.1 |
| [Xenopus laevis] | | |
| >gb\|AAH77577.1 | | |
| aquaporin 3 (Gill blood group) | 94 | NP_001016845.1 |
| [Xenopus (Silurana) | | |
| tropicalis] >emb\|CAJ82459.1 | | |
| PREDICTED: aquaporin-3 [Gorilla | 78 | XP_004047976.1 |
| gorilla gorilla] | | |
| PREDICTED: aquaporin-3-like | 65 | XP_003975282.1 |
| [Takifugu rubripes] | | |
| aquaporin [Sparus aurata] | 50 | AAR13054.1 |

TABLE 3 e)
ZYRO0E01210p from Zygosaccharomyces rouxii and
proteins with a similar amino acid sequence identity.

| | Identity | Accession |
|---|---|---|
| Description | (%) | number |
| ZYRO0E01210p [Zygosaccharomyces rouxii] | 100 | XP_002498999.1 |
| >emb\|CAR30744.1\| ZYRO0E01210p | | |
| [Zygosaccharomyces rouxii] | | |
| BN860_18536g1_1 [Zygosaccharomyces | 82 | CDF87965.1 |
| bailii CLIB 213] | | |
| hypothetical protein TDEL_0B07220 | 66 | XP_003680062.1 |
| [Torulaspora delbrueckii] >emb\|CCE90851.1\| | | |
| hypothetical protein | | |
| TDEL_0B07220 [Torulaspora delbrueckii] | | |
| Stl1p [Saccharomyces cerevisiae S288c] | 66 | NP_010825.3 |
| >sp\|P39932.2 | | |
| sugar transporter STL1 [Candida | 64 | EEQ46634.1 |
| albicans WO-1] | | |
| monosaccharide transporter | 45 | XP_003193210.1 |
| [Cryptococcus gattii WM276] | | |
| >gb\|ADV21423.1 | | |

As indicated above, the recombinant yeast preferably comprises glycerol-proton symporter activity. That is, the recombinant yeast preferably comprises one or more nucleic acid sequences encoding for a heterologous protein having glycerol-proton symporter activity. An example of such glycerol-proton symporter proteins are STL1 proteins.

More preferably the recombinant yeast comprises one or more glucose-tolerant nucleic acid sequence(s) encoding one or more heterologous protein(s) with glycerol-proton symporter activity.

Hence preferably the recombinant yeast comprises a gene encoding for a protein comprising an amino acid sequence represented by SEQ ID NO: 1, 2, 3, 4 or 5 herein or a functional homologue of respectively SEQ ID NO:1, 2, 3, 4 or 5 herein having a sequence identity of at least 60%, at least 65%, at least 70%, at least 75%, at least 80%, at least 85%, at least 90%, at least 95%, at least 98% or at least 99% with respectively SEQ ID No:1, 2, 3, 4 or 5 herein.

Still more preferably the recombinant yeast comprises one or more glucose-tolerant STL genes. Most preferably the recombinant yeast comprises one or more nucleic acid sequence(s) encoding for a heterologous protein represented by SEQ ID NO: 5 or a functional homologue of SEQ ID NO:5 having an amino acid sequence with at least 50, 55, 60, 65, 70, 75, 80, 85, 90, 95, 98, 99% amino acid sequence identity with SEQ ID No:5, suitably allowing the recombinant yeast to functionally express such proteins.

The nucleic acid sequence (e.g. the gene) encoding for the glycerol-uptake protein may suitably be incorporated in the genome of the recombinant yeast, for example as described in the examples of WO2015/028583, herein incorporated by reference.

Reduction or Disruption of the Activity of One or More Homologous Protein(s) with Glycerol-Efflux Activity

In the recombinant yeast, the activity of one, or more, homologous protein(s) with glycerol-efflux activity is suitably reduced, downregulated, inhibited and/or eliminated. One skilled in the art will understand that such reduction, downregulation, inhibition and/or elimination as described herein is suitable as compared to the corresponding parent or wild-type yeast. By glycerol-efflux activity is herein understood the activity of transporting glycerol from the cell into the surrounding medium, for example into the fermentation medium.

By a homologous protein is herein understood a protein that is native to the yeast.

Nowadays many glycerol transporters (such as channels, facilitators and symporters) have been identified, characterized biochemically and the corresponding genes have been cloned.

It has now surprisingly been found that the combination of introducing glycerol uptake activity with a heterologous protein as described above and reducing, downregulating, inhibiting and/or eliminating glycerol-efflux activity of one or more homologous protein(s), can surprisingly improve the amount and rate of the conversion of arabinose.

As also indicated in WO2015/028583, in case of S. cerevisiae, four different genes have been implicated with glycerol transport, FPS1, GUP1, GUP2 and STL1. In WO2015/028583, a more detailed description of protein function of proteins encoded by FPS1, GUP1, GUP2 and STL1 is provided, as taken from www.yeastgenome.org)

In recombinant yeast in the inventions preferably the activity of one or more homologous aquaglyceroporines with glycerol-efflux activity is reduced, downregulated, inhibited and/or eliminated. Aquaglyceroporins are membrane channels that mediate fluxes of water and small solute molecules.

More preferably the activity of one or more homologous aquaglyceroporines with glycerol-efflux activity is reduced, downregulated or eliminated.

That is, preferably the genes or nucleic acid sequence's encoding for such an aquaglyceroporine are genetically modified or knocked-out as compared to the parent yeast (or wild-type yeast). This in turn may suitably result in the deletion or disruption of the native aquaglyceroporine protein from the yeast.

Most preferably the activity of the protein encoded by FPS1 (fps1), or a similar protein, is reduced, downregulated, inhibited and/or eliminated. Examples of nucleic acid sequences for FPS1 such as the FPS1 gene from e.g. S. cerevisiae can be found in Van Aelst et al. (1991, EMBO J. vol. 10, pages 2095-2104), and orthologues thereof from other yeasts including Kluyveromyces lactis, Kluyveromyces marxianus and Zygosaccharomyces rouxii are described by Neves et al. (2004, FEMS Yeast Res. Vol. 5, pages 51-62), herein incorporated by reference.

According to WO2015/023989, fps1 is a channel protein located in the plasma membrane of yeasts such as S. cerevisiae, that controls the accumulation and release of glycerol in yeast osmoregulation. WO2015/023989 indicated that null mutants of this strain accumulate large amounts of intracellular glycerol and hence grow much slower than wild-type, and consume the sugar substrate at a slower rate. (referring to Tamas, M. J., et al., Mol. Microbiol. 57: 1087-1004 (1999). Surprisingly, however, inventors have now found that for the process and recombinant yeast according to the present invention, the rate of conversion of arabinose into ethanol has actually improved.

In order to reduce, downregulate, inhibit or eliminate the activity of one or more homologous protein(s) with glycerol-efflux activity, suitably aquaglyceroporins and preferably proteins encoded by the FPS1 gene, the recombinant yeast may comprise one or more genetic modifications that reduce the glycerol-efflux activity in the host cell, that is, in the native yeast cell.

For example, glycerol-efflux activity can be reduced in the host cell by one or more genetic modifications that reduce the expression of or inactivates the FPS1 gene encoding for the fps1 protein. Preferably, the genetic modification(s) reduce or inactivate the expression of each endogenous copy of the FPS1 gene (herein called FPS1 deletion). Preferably, the gene is inactivated by deletion of at least part of the gene or by disruption of the gene, whereby in this context the term gene also includes any non-coding sequence up- or down-stream of the coding sequence, the (partial) deletion or inactivation of which results in a reduction of expression of glycerol-efflux activity in the host cell.

Preferably the activity of one or more homologous protein(s) with glycerol-efflux activity is reduced, downregulated, inhibited and/or eliminated by whole or partial disruption or deletion of one or more gene(s) encoding for such homologous protein(s), such as exemplified above for the FPS1 deletion. The activity of one or more homologous protein(s) with glycerol-efflux activity may, however, also be reduced, downregulated, inhibited and/or eliminated in a less direct manner, for example by destabilizing the protein or by inhibiting expression of the gene. For example, an aquaglyceroporin can be destabilized by direct MAPK (mitogen-activated protein kinase) phosphorylation as described by Mollapour et al. 2007. Surprisingly, whilst Mollapour et al describe that for the yeast described in their article disruption of Fps 1 downregulates the passive influx of undissociated acetic acid into the cell, examples of the present invention illustrate that for the yeast cell according to the present invention, conversion of acetic acid into ethanol is actually improved.

Alternatively, rather than deleting or disrupting one or more homologous genes encoding for protein(s) with glycerol-efflux activity, heterologous genes from other organisms or artificial nucleic acid sequences can be inserted to replace such homologous genes to downregulate glycerol-efflux activity.

Most preferably the recombinant yeast comprises a genetic modification (i.e. as compared to the parent or wild-type yeast) leading to the deletion or disruption of a homologous protein comprising an amino acid sequence represented by SEQ ID NO: 14 herein or a functional homologue of SEQ ID NO:14 herein having a sequence identity of at least 60%, at least 65%, at least 70%, at least 75%, at least 80%, at least 85%, at least 90%, at least 95%, at least 98% or at least 99% with SEQ ID No:14 herein.

Glycerol Dehydrogenase

In addition to the glucose and arabinose, the carbon source composition may further comprise glycerol. Glycerol is a carbon source having a similar carbon content as glucose and arabinose. However, glycerol is not a sugar but a so-called polyol.

The recombinant yeast suitably comprises glycerol conversion capacity, herein also referred to as glycerol conversion activity. By glycerol conversion capacity or glycerol conversion activity, is herein understood that the recombinant yeast comprises one or more nucleic acid sequences encoding for one or more, preferably heterologous, protein(s) capable of converting glycerol, i.e. having glycerol conversion activity. The presence of such, preferably exogenous, nucleic acid sequences allows the recombinant yeast to functionally express such a protein. Preferably such one or more heterologous protein(s) capable of converting glycerol are part of a so-called glycerol conversion pathway, allowing the recombinant yeast to convert glycerol into dihydroxyacetone, which dihydroxyacetone may subsequently be converted by a pathway native to the yeast into ethanol.

The recombinant yeast preferably comprises, one, two or more copies of an exogenous gene encoding for one or more heterologous protein(s) having glycerol dehydrogenase activity. Hence allowing the recombinant yeast to functionally express such a protein.

A protein with glycerol dehydrogenase activity is often also referred to as a glycerol dehydrogenase. The glycerol dehydrogenase can use NAD⁺ or NADP⁺ as acceptor. Preferably the recombinant yeast comprises one or more, preferably exogenous, nucleic acid sequences encoding for one or more, preferably heterologous, proteins having NAD+-dependent glycerol dehydrogenase activity. That is, preferably the glycerol dehydrogenase in the recombinant yeast is a NAD⁺-dependent glycerol dehydrogenase (EC1.1.1.6), also referred to as NAD⁺-linked glycerol dehydrogenase.

NAD+-dependent glycerol dehydrogenase is an enzyme that catalyzes the chemical reaction (I):

glycerol+NAD⁺↔glyceron+NADH+H+ (I)

Other names in common use for this enzyme include glycerin dehydrogenase and glycerol:NAD+2-oxidoreductase. Another name in common use for glyceron is dihydroxyacetone.

The glycerol dehydrogenase encoded by the endogenous yeast GCY1 gene appears to be specific for the cofactor NADP+(EC 1.1.1.72) as opposed to NAD+(EC 1.1.1.6). Yeasts such as S. cerevisiae appear to lack NAD+-dependent glycerol dehydrogenase activity (EC 1.1.1.6) (see e.g. KEGG pathway 00561). Hence, preferably the recombinant yeast comprises one or more nucleic acid sequences encoding for one or more heterologous proteins having NAD+-dependent glycerol dehydrogenase activity. The recombinant yeast preferably comprises a genetic modification that introduces NAD⁺-dependent glycerol dehydrogenase activity in the yeast cell. Such may allow for the expression of an NAD⁺-dependent glycerol dehydrogenase that is heterologous to the recombinant yeast.

Preferably, the nucleic acid sequence for expression of a heterologous glycerol dehydrogenase in the recombinant yeast is a nucleic acid sequence encoding a bacterial glycerol dehydrogenase using NAD+ as cofactor (EC 1.1.1.6).

A suitable example of a bacterial NAD⁺-dependent glycerol dehydrogenase for expression in the recombinant yeast in the invention is the NAD⁺-dependent glycerol dehydrogenase expressed in E. coli, e.g. the gldA gene from E. coli described by Truniger and Boos (1994, J Bacteriol. 176(6):1796-1800), the expression of which in yeast has already been reported (Lee and Dasilva, 2006, Metab Eng. 8(1):58-65).

More preferably the nucleic acid sequence encoding a heterologeous glycerol dehydrogenase in the recombinant yeast is a nucleic acid sequence encoding for an amino acid sequence with at least 45, 50, 60, 65, 70, 75, 80, 85, 90, 95, 98, 99% amino acid sequence identity with gldA from Escherichia coli, gldA from Klebsiella pneumoniae, gldA from Enterococcus aerogenes, or gldA from Yersinia aldovae.

Most preferably, the nucleic acid sequence encoding a heterologous glycerol dehydrogenase comprises a nucleic acid sequence coding for an amino acid sequence with at least 45, 50, 60, 65, 70, 75, 80, 85, 90, 95, 98, 99% amino acid sequence identity with SEQ ID NO: 7 or a nucleic acid sequence coding for an amino acid sequence having one or several substitutions, insertions and/or deletions as compared to SEQ ID NO: 7. In a preferred embodiment a codon-optimised (see above) nucleic acid sequence encoding the heterologous glycerol dehydrogenase is overexpressed, such as e.g. a codon-optimised nucleic acid sequence encoding the amino acid sequence of the glycerol dehydrogenase of SEQ ID NO: 7.

For overexpression of the nucleic acid sequence encoding the glycerol dehydrogenase, the nucleic acid sequence (to be overexpressed) can be placed in an expression construct wherein it is operably linked to suitable expression regulatory regions/sequences to ensure overexpression of the glycerol dehydrogenase enzyme upon transformation of the expression construct into the recombinant yeast. Suitable promoters for (over)expression of the nucleic acid sequence coding for the enzyme having glycerol dehydrogenase activity include promoters that are preferably insensitive to catabolite (glucose) repression, that are active under anaerobic conditions and/or that preferably do not require xylose or arabinose for induction. Examples of such promoters are given above. Expression of the nucleic acid sequence in the recombinant yeast preferably produces a specific NAD⁺-linked glycerol dehydrogenase activity of at least 0.2, 0.5, 1.0, 2.0, or 5.0 U min⁻¹ (mg protein)⁻¹, determined in cell extracts of the transformed yeast cells at 30° C.

Further examples of suitable glycerol dehydrogenases are listed in table 4(a) to 4(d). At the top of each table a specific glycerol dehydrogenase is indicated, such as for example in Table 4(a) glycerol dehydrogenase originating from E. coli (gIdA) is listed. For the other examples in each table the amino acid sequence identity with the first listed example is indicated.

TABLE 4 (a)
gldA from Escherichia coli and glycerol dehydrogenases
with similar amino acid sequence identity.

| | Identity | Accession |
|---|---|---|
| Description | (%) | number |
| glycerol dehydrogenase, NAD [Escherichia | 100 | NP_418380.4 |
| coli str. K-12 substr MG1655] | | |
| glycerol dehydrogenase [Escherichia coli | 99 | YP_002331714.1 |
| O27:H6 str. E2348/69] | | |
| glycerol dehydrogenase [Citrobacter youngae] | 94 | WP_006686227.1 |
| glycerol dehydrogenase [Citrobacter freundii] | 92 | WP_003840533.1 |

TABLE 4 (b)
gldA from Klebsiella pneumoniae and glycerol
dehydrogenases with similar amino acid sequence identity.

| | Identity | Accession |
|---|---|---|
| Description | (%) | number |
| glycerol dehydrogenase [Klebsiella | 100 | YP_002236495.1 |
| pneumoniae 342] | | |
| glycerol dehydrogenase [Citrobacter freundii] | 93 | WP_003024745.1 |
| Glycerol dehydrogenase (EC 1.1.1.6) | 92 | YP_004590977.1 |
| [Enterobacter aerogenes EA1509E] | | |
| glycerol dehydrogenase [Escherichia coli] | 91 | WP_016241524.1 |
| glycerol dehydrogenase [Yersinia aldovae] | 74 | WP_004701845.1 |
| glycerol dehydrogenase [Enterobacteriaceae | 61 | WP_017375113.1 |
| bacterium LSJC7] | | |
| glycerol dehydrogenase [Citrobacter youngae] | 60 | WP_006686227.1 |

TABLE 4 (c)
gldA from Enterococcus aerogenes and glycerol
dehydrogenases with similar amino acid sequence identity.

| | Identity | Accession |
|---|---|---|
| Description | (%) | number |
| glycerol dehydrogenase [Enterobacter | 100 | YP_004591726.1 |
| aerogenes KCTC 2190] | | |
| Glycerol dehydrogenase (EC 1.1.1.6) | 99 | YP_007390021.1 |
| [Enterobacter aerogenes EA1509E] | | |
| glycerol dehydrogenase [Klebsiella | 92 | WP_004203683.1 |
| pneumoniae] | | |
| glycerol dehydrogenase [Escherichia coli] | 88 | WP_001322519.1 |
| glycerol dehydrogenase [Enterobacter | 87 | YP_003615506.1 |
| cloacae subsp. cloacae ATCC 13047] | | |

TABLE 4 (d)
gldA from Yersinia aldovae and glycerol dehydrogenases
with similar amino acid sequence identity.

| | Identity | Accession |
|---|---|---|
| Description | (%) | number |
| glycerol dehydrogenase [Yersinia aldovae] | 100 | WP_004701845.1 |
| glycerol dehydrogenase [Yersinia intermedia] | 95 | WP_005189747.1 |
| glycerol dehydrogenase [Serratia liquefaciens | 81 | YP_008232202.1 |
| ATCC 27592] | | |
| glycerol dehydrogenase [Escherichia coli] | 76 | WP_016241524.1 |
| hypothetical protein EAE_03845 | 75 | YP_004590977.1 |
| [Enterobacter aerogenes KCTC 2190] | | |
| glycerol dehydrogenase [Aeromonas | 65 | WP_017410769.1 |
| hydrophila] | | |

### Dihydroxyacetone Kinase

Preferably the recombinant yeast further comprises one or more nucleic acid sequence(s) encoding one or more protein(s) with dihydroxyacetone kinase activity.

A dihydroxyacetone kinase is an enzyme that catalyzes the chemical reaction (II):

ATP+glycerone↔ADP+glycerone phosphate (II)

Other names in common use for this enzyme include glycerone kinase, ATP:glycerone phosphotransferase and (phosphorylating) acetol kinase. Another name in common use for glyceron is dihydroxyacetone.

The recombinant yeast may already comprise homologous dihydroxyacetone kinase, i.e. native to the yeast. Transcriptome data has shown that the endogenous DAK1 dihydroxyacetone kinase is already expressed at high levels in S. cerevisiae. A further increase of dihydroxyacetone kinase activity in the cells of the invention may therefore not be strictly necessary. However, in a preferred, embodiment, for optimal conversion rates, the yeast cell of the invention may comprise a genetic modification that increases the specific activity of dihydroxyacetone kinase in the cell. Preferably such a genetic modification causes overexpression of a dihydroxyacetone kinase, e.g. by overexpression of a nucleic acid sequence encoding a dihydroxyacetone kinase.

The nucleic acid sequence encoding the dihydroxyacetone kinase may be endogenous to the cell or may be a nucleic acid sequence encoding dihydroxyacetone kinase that is exogenous to the cell. Nucleotide sequences that may be used for overexpression of dihydroxyacetone kinase in the cells of the invention are e.g. the dihydroxyacetone kinase genes from S. cerevisiae (DAK1) and (DAK2) as e.g. described by Molin et al. (2003, J. Biol. Chem. 278:1415-1423).

More preferably the nucleic acid sequence encoding a heterologeous glycerol dehydrogenase in the yeast cell of the invention is a nucleic acid sequence encoding for an amino acid sequence with at least 45, 50, 60, 65, 70, 75, 80, 85, 90, 95, 98, 99% amino acid sequence identity with DAK1 from S. cerevisiae, dhaK from Klebsiella pneumoniae, DAK1 from Yarrowia lipolytica, or DAK1 from Schizosaccharomyces pombe. Still more preferably the recombinant yeast comprises DAK1 and/or DAK 2 of S. cerevisiae.

Most preferably, the nucleic acid sequence encoding the dihydroxyacetone kinase comprises an amino acid sequence with at least 45, 50, 60, 65, 70, 75, 80, 85, 90, 95, 98, 99% amino acid sequence identity with at least one of SEQ ID NO's: 8 (DAK1 S. cerevisiae) and 9 (DAK2 S. cerevisiae).

In a preferred embodiment a codon-optimised (see above) nucleic acid sequence encoding the dihydroxyacetone kinase is overexpressed, such as e.g. a codon optimised nucleic acid sequence encoding the dihydroxyacetone kinase of SEQ ID NO: 8 or a codon optimised nucleic acid sequence encoding the dihydroxyacetone kinase of SEQ ID NO: 9. A preferred nucleic acid sequence for overexpression of a dihydroxyacetone kinase is a nucleic acid sequence encoding a dihydroxyacetone kinase comprises an amino acid sequence with at least 45, 50, 60, 65, 70, 75, 80, 85, 90, 95, 98, 99% amino acid sequence identity with at least one of SEQ ID NO: 8 (S. cerevisiae (DAK1) or having one or several substitutions, insertions and/or deletions as compared to SEQ ID NO: 8.

Nucleotide sequences that may be used for overexpression of a heterologous dihydroxyacetone kinase in the cells of the invention are e.g. sequences encoding bacterial dihydroxyacetone kinases such as the dhaK gene from Citrobacter freundii e.g. described by Daniel et al. (1995, J. Bacteriol. 177:4392-4401). Preferably, the nucleic acid sequence encoding a heterologous dihydroxyacetone kinase comprises a nucleic acid sequence coding for an amino acid sequence with at least 45, 50, 60, 65, 70, 75, 80, 85, 90, 95, 98, 99% amino acid sequence identity with SEQ ID NO: 8 or a nucleic acid sequence coding for an amino acid sequence having one or several substitutions, insertions and/or deletions as compared to SEQ ID NO: 8. In a preferred embodiment a codon-optimised (see above) nucleic acid sequence encoding the heterologous dihydroxyacetone kinase is overexpressed, such as e.g. a codon optimised nucleic acid sequence encoding the amino acid sequence of the dihydroxyacetone kinase of SEQ ID NO: 8.

For overexpression of the nucleic acid sequence encoding the dihydroxyacetone kinase, the nucleic acid sequence (to be overexpressed) is placed in an expression construct wherein it is operably linked to suitable expression regulatory regions/sequences to ensure overexpression of the dihydroxyacetone kinase enzyme upon transformation of the expression construct into the yeast cell of the invention (see above). Suitable promoters for (over)expression of the nucleic acid sequence coding for the enzyme having dihydroxyacetone kinase activity include promoters that are preferably insensitive to catabolite (glucose) repression, that are active under anaerobic conditions and/or that preferably do not require xylose or arabinose for induction. Examples of such promoters are given above. In the cells of the invention, a dihydroxyacetone kinase to be overexpressed is preferably overexpressed by at least a factor 1.1, 1.2, 1.5, 2, 5, 10 or 20 as compared to a strain which is genetically identical except for the genetic modification causing the overexpression. Preferably, the dihydroxyacetone kinase is overexpressed under anaerobic conditions by at least a factor 1.1, 1.2, 1.5, 2, 5, 10 or 20 as compared to a strain which is genetically identical except for the genetic modification causing the overexpression. It is to be understood that these levels of overexpression may apply to the steady state level of the enzyme's activity (specific activity in the cell), the steady state level of the enzyme's protein as well as to the steady state level of the transcript coding for the enzyme in the cell. Overexpression of the nucleic acid sequence in the yeast cell produces a specific dihydroxyacetone kinase activity of at least 0.002, 0.005, 0.01, 0.02 or 0.05 U min⁻¹ (mg protein)⁻¹, determined in cell extracts of the transformed yeast cells at 30° C.

Further examples of suitable dihydroxyacetone kinases are listed in table 5(a) to 5(d). At the top of each table a specific dihydroxyacetone kinase is indicated, such as for example in Table 5(a) DAK1 from Saccharomyces cerevisiae is listed. For the other examples in each table the amino acid sequence identity with the first listed example is indicated.

TABLE 5 (a)
DAK1 from Saccharomyces cerevisiae and dihydroxyacetone
kinases with similar amino acid sequence identity.

| | Identity | Accession |
|---|---|---|
| Description | (%) | number |
| Dak1p [Saccharomyces cerevisiae S288c] | 100 | NP_013641.1 |
| dihydroxyacetone kinase [Saccharomyces | 99 | EDN64325.1 |
| cerevisiae YJM789] | | |
| DAK1-like protein [Saccharomyces | 95 | EJT44075.1 |
| kudriavzevii IFO 1802] | | |
| ZYBA0S11-03576g1_1 [Zygosaccharomyces | 77 | CDF91470.1 |
| bailii CLIB 213] | | |
| hypothetical protein [Kluyveromyces lactis | 70 | XP_451751.1 |
| NRRL Y-1140] | | |
| hypothetical protein [Candida glabrata CBS 138] | 63 | XP_449263.1 |
| Dak2p [Saccharomyces cerevisiae S288c] | 44 | NP_116602.1 |

TABLE 5b
dhaK from Klebsiella pneumoniae and dihydroxyacetone kinases
with similar amino acid sequence identity.

| | Identity | Accession |
|---|---|---|
| Description | (%) | number |
| dihydroxyacetone kinase subunit DhaK | 100 | YP_002236493.1 |
| [Klebsiella pneumoniae 342] | | |
| dihydroxyacetone kinase subunit K | 99 | WP_004149886.1 |
| [Klebsiella pneumoniae] | | |
| dihydroxyacetone kinase subunit K | 96 | WP_020077889.1 |
| [Enterobacter aerogenes] | | |
| dihydroxyacetone kinase subunit DhaK | 88 | YP_002407536.1 |
| [Escherichia coli IAI39] | | |
| dihydroxyacetone kinase, DhaK subunit | 87 | WP_001398949.1 |
| [Escherichia coli] | | |

TABLE 5c
DAK1 from Yarrowia lipolytica and dihydroxyacetone kinases with
similar amino acid sequence identity.

| | Identity | Accession |
|---|---|---|
| Description | (%) | number |
| YALIOFO9273p [Yarrowia lipolytica] | 100 | XP_505199.1 |
| dihydroxyacetone kinase [Schizosaccharomyces | 46 | AAC83220.1 |
| pombe] | | |
| dihydroxyacetone kinase Dak1 | 45 | NP_593241.1 |
| [Schizosaccharomyces pombe 972h-] | | |
| dihydroxyacetone kinase [Saccharomyces | 44 | EDV12567.1 |
| cerevisiae RM11-1a] | | |
| Dak2p [Saccharomyces cerevisiae JAY291] | 44 | EEU04233.1 |
| BN860_19306g1_1 [Zygosaccharomyces bailii | 44 | CDF87998.1 |
| CLIB 213] | | |
| Dak1p [Saccharomyces cerevisiae | 42 | EIW08612.1 |
| CEN.PK113-7D] | | |

TABLE 5d
DAK1 from Schizosaccharomyces pombe and dihydroxyacetone kinases
with similiar amino acid sequence identity.

| | Identity | Accession |
|---|---|---|
| Description | (%) | number |
| dihydroxyacetone kinase Dak1 | 100 | NP_593241.1 |
| [Schizosaccharomyces pombe 972h-] | | |
| putative di hydroxyacetone kinase protein | 48 | EMR88164.1 |
| [Botryotinia fuckeliana BcDW1] | | |
| Dihydroxyacetone kinase 1 [Fusarium | 48 | ENH64704.1 |
| oxysporum f. sp. cubense race 1] | | |
| Dak1p [Saccharomyces cerevisiae | 46 | EIW08612.1 |
| CEN.PK113-7D] | | |
| Dak2p [Saccharomyces cerevisiae JAY291] | 44 | EEU04233.1 |
| di hydroxyacetone kinase [Exophiala | 42 | EHY55064.1 |
| dermatitidis NIH/UT8656] | | |

### Co-Factor Balance

Glycerol production under anaerobic conditions is primarily linked to the NAD+/NADH co-factor balance of the yeast cell. During anaerobic growth of for example S. cerevisiae, sugar dissimilation occurs via alcoholic fermentation. In this process, the NADH formed in a glycolytic glyceraldehyde-3-phosphate dehydrogenase reaction can be reoxidized by converting acetaldehyde, formed by decarboxylation of pyruvate to ethanol, via NAD+-dependent alcohol dehydrogenase. The fixed stoichiometry of this redox-neutral dissimilatory pathway causes problems when a net reduction of NAD+ to NADH occurs elsewhere in metabolism.

In conventional processes under anaerobic conditions, NADH reoxidation in S. cerevisiae is strictly dependent on reduction of sugar to glycerol. Glycerol formation is initiated by reduction of the glycolytic intermediate dihydroxyacetone phosphate to glycerol 3-phosphate, a reaction catalyzed by NADH-dependent glycerol 3-phosphate dehydrogenase. Subsequently, the glycerol 3-phosphate formed in this reaction is hydrolysed by glycerol-3-phosphatase to yield glycerol and inorganic phosphate. When such glycerol is not transported out of the yeast cell, but is converted by for example NAD+dependent glycerol dehydrogenase into dihydroxyacetone, such may result in a NAD+/NADH cofactor imbalance.

In the yeast cell according to the invention, such a potential NAD+/NADH cofactor imbalance is preferably resolved by introducing one or more nucleic acid sequence(s) encoding for a heterologous NADH-oxidizing enzyme or a NADH-oxidizing pathway into the yeast cell. Hence, preferably the recombinant yeast further comprises one or more nucleic acid sequence(s) encoding for a heterologous NADH-oxidizing enzyme or enzymatic pathway.

Preferably the carbon source composition further comprises acetic acid, or a salt thereof; and preferably the recombinant yeast further comprises a gene encoding for a heterologous protein having acetyl-Coenzyme A synthetase activity; and/or a gene encoding for a heterologous protein having acetylating acetaldehyde dehydrogenase activity, and preferably the acetic acid, or the salt thereof, is converted to ethanol.

More preferably the recombinant yeast comprises one or more, suitably exogenous, nucleic acid sequence(s) encoding for one or more protein(s) having heterologous acetylating acetaldehyde dehydrogenase activity;

Preferably the yeast cell according to the invention comprises one or more exogenous nucleic acid sequences coding for a protein with the ability to reduce acetylCoA into acetaldehyde, which gene confers to the cell the ability to convert acetylCoA (and/or acetic acid) into ethanol. An enzyme with the ability to reduce acetylCoA into acetaldehyde is herein understood as an enzyme which catalyzes the reaction (ACDH; EC 1.2.1.10):

acetaldehyde+NAD⁺+Coenzyme A [symbol] acetyl-Coenzyme A+NADH+H⁺. (III)

Thus, the enzyme catalyzes the conversion of acetylCoA into acetaldehyde (and vice versa) and is also referred to as an (acetylating NAD-dependent) acetaldehyde dehydrogenase or an acetyl-CoA reductase. The enzyme may be a bifunctional enzyme which further catalyzes the conversion of acetaldehyde into ethanol (and vice versa; see below).

For convenience we shall refer herein to an enzyme having at least the ability to reduce acetylCoA into either acetaldehyde or ethanol as an “acetaldehyde dehydrogenase”. It is further understood herein that the cell has endogenous alcohol dehydrogenase activities which allow the cell, being provided with acetaldehyde dehydrogenase activity, to complete the conversion of acetyl-CoA into ethanol. Further the cell has endogenous or exogenous acetyl-CoA synthetase, which allows the cell, being provided with acetaldehyde dehydrogenase activity, to complete the conversion of acetic acid (via acetyl-CoA) into ethanol.

The exogenous gene may encode for a monofunctional enzyme having only acetaldehyde dehydrogenase activity (i.e. an enzyme only having the ability to reduce acetylCoA into acetaldehyde) such as e.g. the acetaldehyde dehydrogenase encoded by the E. coli mhpF gene. Suitable examples of prokaryotes comprising monofunctional enzymes with acetaldehyde dehydrogenase activity are provided in Table 6. The amino acid sequences of these monofunctional enzymes are available in public databases and can be used by the skilled person to design codon-optimized nucleotide sequences coding for the corresponding monofunctional enzyme.

TABLE 6
Suitable enzymes with acetaldehyde dehydrogenase activity and
identity to E.coli mhpF

| | Amino acid |
|---|---|
| Sequence and Organism | identity (%) |
| Escherichia coli str. K12 substr. MG 1655 | 100% |
| Shigella sonnei | 100% |
| Escherichia coli IAI39 | 99% |
| Citrobacter youngae ATCC 29220 | 93% |
| Citrobacter sp. 30_2 | 92% |
| Klebsiella pneumoniae 342) | 87% |
| Klebsiella variicola | 87% |
| Pseudomonas putida | 81% |
| Ralstonia eutropha JMP134 | 82% |
| Burkholderia sp. H160 | 81% |
| Azotobacter vinelandii DJ | 79% |
| Ralstonia metallidurans CH34 | 70% |
| Xanthobacter autotrophicus Py2 | 67% |
| Burkholderia cenocepacia J2315 | 68% |
| Frankia sp. EAN1pec | 67% |
| Polaromonas sp. JS666 | 68% |
| Burkholderia phytofirmans PsJN | 70% |
| Rhodococcus opacus B4 | 64% |

In an embodiment, recombinant yeast comprises an exogenous gene coding for a bifunctional enzyme with acetaldehyde dehydrogenase and alcohol dehydrogenase activity, which gene confers to the cell the ability to convert acetylCoA into ethanol. The advantage of using a bifunctional enzyme with acetaldehyde dehydrogenase and alcohol dehydrogenase activities as opposed to separate enzymes for each of the acetaldehyde dehydrogenase and alcohol dehydrogenase activities, is that it allows for direct channeling of the intermediate between enzymes that catalyze consecutive reactions in a pathway offers the possibility of an efficient, exclusive, and protected means of metabolite delivery. Substrate channeling thus decreases transit time of intermediates, prevents loss of intermediates by diffusion, protects labile intermediates from solvent, and forestalls entrance of intermediates into competing metabolic pathways. The bifunctional enzyme therefore allows for a more efficient conversion of acetylCoA into ethanol as compared to the separate acetaldehyde dehydrogenase and alcohol dehydrogenase enzymes. A further advantage of using the bifunctional enzyme is that it may also be used in cells having little or no alcohol dehydrogenase activity under the condition used, such as e.g. anaerobic conditions and/or conditions of catabolite repression.

Bifunctional enzymes with acetaldehyde dehydrogenase and alcohol dehydrogenase activity are known in the art prokaryotes and protozoans, including e.g. the bifunctional enzymes encoded by the Escherichia coli adhE and Entamoeba histolytica ADH2 genes (see e.g. Bruchaus and Tannich, 1994, J. Biochem. 303: 743-748; Burdette and Zeikus, 1994, J. Biochem. 302: 163-170; Koo et al., 2005, Biotechnol. Lett. 27: 505-510; Yong et al., 1996, Proc Natl Acad Sci USA, 93: 6464-6469). Bifunctional enzymes with acetaldehyde dehydrogenase and alcohol dehydrogenase activity are larger proteins consisting of around 900 amino acids and they are bifunctional in that they exhibit both acetaldehyde dehydrogenase (ACDH; EC 1.2.1.10) and alcohol dehydrogenase activity (ADH; EC 1.1.1.1). The E. coli adhE and Entamoeba histolytica ADH2 show 45% amino acid identity.

TABLE 7
Suitable bifunctional enzymes with acetaldehyde dehydrogenase
and alcohol dehydrogenase activity and identity to E.coli adhE

| | Amino acid |
|---|---|
| Sequence and Organism | identity (%) |
| Escherichia coli O157:H7 str. Sakai | 100% |
| Shigella sonnei | 100% |
| Shigella dysenteriae 1012 | 99% |
| Klebsiella pneumoniae 342 | 97% |
| Enterobacter sp. 638 | 94% |
| Yersinia pestis biovar Microtus str. 91001 | 90% |
| Serratia proteamaculans 568 | 90% |
| Pectobacterium carotovorum WPP14 | 90% |
| Sodalis glossinidius str. 'morsitans' | 87% |
| Erwinia tasmaniensis Et1/99 | 86% |
| Aeromonas hydrophila ATCC 7966 | 81% |
| Vibrio vulnificus YJ016] | 76% |

TABLE 8
Suitable bifunctional enzymes with acetaldehyde dehydrogenase
and alcohol dehydrogenase activity and identity to Entamoeba
histolytica ADH2

| | Amino acid |
|---|---|
| Sequence and Organism | identity (%) |
| Entamoeba histolytica HM-1:IMSS | 99% |
| Entamoeba dispar SAW760 | 98% |
| Mollicutes bacterium D7 | 65% |
| Fusobacterium mortiferum ATCC 9817 | 64% |
| Actinobacillus succinogenes 130Z | 63% |
| Pasteurella multocida Pm70 | 62% |
| Mannheimia succiniciproducens MBEL55E | 61% |
| Streptococcus sp. 2_1_36FAA] | 61% |

For expression of the nucleotide sequence encoding the bifunctional enzyme having acetaldehyde dehydrogenase and alcohol dehydrogenase activities, or the enzyme having acetaldehyde dehydrogenase activity, the nucleotide sequence (to be expressed) is placed in an expression construct wherein it is operably linked to suitable expression regulatory regions/sequences to ensure expression of the enzyme upon transformation of the expression construct into the cell of the invention (see above). Suitable promoters for expression of the nucleotide sequence coding for the enzyme having the bifunctional enzyme having acetaldehyde dehydrogenase and alcohol dehydrogenase activities, or the enzyme having acetaldehyde dehydrogenase activity include promoters that are preferably insensitive to catabolite (glucose) repression, that are active under anaerobic conditions and/or that preferably do not require xylose or arabinose for induction. Examples of such promoters are given above.

Preferably, the nucleotide sequence encoding the bifunctional enzyme having acetaldehyde dehydrogenase and alcohol dehydrogenase activities, or the enzyme having acetaldehyde dehydrogenase activity is adapted to optimize its codon usage to that of the cell in question (as described above).

Preferably the recombinant yeast comprises an exogenous gene encoding for E. coli adhE, allowing such protein to be functionally expressed therein.

Known NAD+-dependent acetylating acetaldehyde dehydrogenases that can catalyse the NADH-dependent reduction of acetyl-Coenzyme A to acetaldehyde may in general be divided in three types of NADH-dependent acetylating acetaldehyde dehydrogenase functional homologues:

1) Bifunctional proteins that catalyse the reversible conversion of acetyl-Coenzyme A to acetaldehyde, and the subsequent reversible conversion of acetaldehyde to ethanol. An example of this type of proteins is the AdhE protein in E. coli (Gen Bank No: NP-415757). AdhE appears to be the evolutionary product of gene fusion. The NH2-terminal region of the AdhE protein is highly homologous to aldehyde:NADH oxidoreductases, whereas the COOH-terminal region is homologous to a family of Fe2+-dependent ethanol:NADH oxidoreductases (Membrillo-Hernandez et al., (2000) J. Biol. Chem. 275: 33869-33875). The E. coli AdhE is subject to metal-catalyzed oxidation and therefore oxygen-sensitive (Tamarit et al. (1998) J. Biol. Chem. 273:3027-32).

2) Proteins that catalyse the reversible conversion of acetyl-Coenzyme A to acetaldehyde in strictly or facultative anaerobic micro-organisms but do not possess alcohol dehydrogenase activity. An example of this type of proteins has been reported in Clostridium kluyveri (Smith et al. (1980) Arch. Biochem. Biophys. 203: 663-675). An acetylating acetaldehyde dehydrogenase has been annotated in the genome of Clostridium kluyveri DSM 555 (GenBank No: EDK33116). A homologous protein AcdH is identified in the genome of Lactobacillus plantarum (GenBank No: NP-784141). Another example of this type of proteins is the said gene product in Clostridium beijerinckii NRRL B593 (Toth et al. (1999) Appl. Environ. Microbiol. 65: 4973-4980, GenBank No: AAD31841).

3) Proteins that are part of a bifunctional aldolase-dehydrogenase complex involved in 4-hydroxy-2-ketovalerate catabolism. Such bifunctional enzymes catalyse the final two steps of the meta-cleavage pathway for catechol, an intermediate in many bacterial species in the degradation of phenols, toluates, naphthalene, biphenyls and other aromatic compounds (Powlowski and Shingler (1994) Biodegradation 5, 219-236). 4-Hydroxy-2-ketovaleraties first converted by 4-hydroxy-2-ketovalerate aldolase to pyruvate and acetaldehyde, subsequently acetaldehyde is converted by acetylating acetaldehyde dehydrogenase to acetyl-CoA. An example of this type of acetylating acetaldehyde dehydrogenase is the DmpF protein in Pseudomonas sp CF600 (GenBank No: CAA43226) (Shingler et al. (1992) J. Bacteriol. 174:71 1-24). The E. coli MphF protein (Ferrandez et al. (1997) J. Bacteriol. 179: 2573-2581, GenBank No: NP-414885) is homologous to the DmpF protein in Pseudomonas sp. CF600.

A suitable nucleic acid sequence may in particular be found in an organism selected from the group of Escherichia, in particular E. coli; Mycobacterium, in particular Mycobacterium marinum, Mycobacterium ulcerans, Mycobacterium tuberculosis; Carboxydothermus, in particular Carboxydothermus hydrogenoformans; Entamoeba, in particular Entamoeba histolytica; Shigella, in particular Shigella sonnei; Burkholderia, in particular Burkholderia pseudomallei, Klebsiella, in particular Klebsiella pneumoniae; Azotobacter, in particular Azotobacter uinelandii; Azoarcus sp; Cupriauidus, in particular Cupriauidus taiwanensis; Pseudomonas, in particular Pseudomonas sp. CF600; Pelomaculum, in particular Pelotomaculum thermopropionicum. Preferably, the nucleic acid sequence encoding the NADH-dependent acetylating acetaldehyde dehydrogenase originates from Escherichia, more preferably from E. coli.

Particularly suitable is an mhpF gene from E. coli, or a functional homologue thereof. This gene is described in Ferrandez et al. (1997) J. Bacteriol. 179:2573-2581. Good results have been obtained with S. cerevisiae, wherein an mhpF gene from E. coli has been incorporated.

In a further advantageous embodiment the nucleic acid sequence encoding an (acetylating) acetaldehyde dehydrogenase is from, in particular Pseudomonas dmpF from Pseudomonas sp. CF600.

In principle, the nucleic acid sequence encoding the NAD+-dependent, acetylating acetaldehyde dehydrogenase may be a wild type nucleic acid sequence. A preferred nucleic acid sequence encodes the NAD+-dependent, acetylating acetaldehyde dehydrogenase represented by SEQ ID NO: 2, SEQ ID NO: 29 in WO2011010923, or a functional homologue of SEQ ID NO: 2 or SEQ ID NO: 29 in WO2011010923. In particular the nucleic acid sequence comprises a sequence according to SEQ ID NO: 1. SEQ ID NO: 28 in WO2011010923 or a functional homologue of SEQ ID NO: 1 or SEQ ID NO: 28 in WO2011010923.

Further, an acetylating acetaldehyde dehydrogenase (or nucleic acid sequence encoding such activity) may in for instance be selected from the group of Escherichia coli adhE, Entamoeba histolytica adh2, Staphylococcus aureus adhE, Piromyces sp.E2 adhE, Clostridium kluyveri EDK33116, Lactobacillus plantarum acdH, and Pseudomonas putida YP 001268189. For sequences of these enzymes, nucleic acid sequences encoding these enzymes and methodology to incorporate the nucleic acid sequence into a host cell, reference is made to WO 20091013159, in particular Example 3, Table 1 (page 26) and the Sequence ID numbers mentioned therein, of which publication Table 1 and the sequences represented by the Sequence ID numbers mentioned in said Table are incorporated herein by reference.

It is further understood, that in a preferred embodiment, that the recombinant yeast has endogenous alcohol dehydrogenase activities which allow the cell, being provided with acetaldehyde dehydrogenase activity, to complete the conversion of acetyl-CoA into ethanol. It is further also preferred that the host cell has endogenous acetyl-CoA synthetase which allow the cell, being provided with acetaldehyde dehydrogenase activity, to complete the conversion of acetic acid (via acetyl-CoA) into ethanol.

Examples of suitable enzymes are adhE of Escherichia coli, acdH of Lactobacillus plantarum, eutE of Escherichia coli, Lin1129 of Listeria innocua and adhE from Staphylococcus aureus. See below tables 11(a) to 11(e) for these enzymes, giving suitable alternative alcohol/acetaldehyde dehydrogenases that are tested in the examples below.

TABLE 11a
adHE from Escherichia coli and proteins with with similar amino
acid sequence identity.

| | Identity | Accession |
|---|---|---|
| Description | (%) | number |
| bifunctional acetaldehyde-CoA/alcohol | 100 | NP_309768.1 |
| dehydrogenase [Escherichia coli O157:H7 | | |
| str. Sakai] | | |
| bifunctional acetaldehyde-CoA/alcohol | 99 | YP_540449.1 |
| dehydrogenase [Escherichia coli UTI89] | | |
| bifunctional acetaldehyde-CoA/alcohol | 95 | YP_001177024.1 |
| dehydrogenase [Enterobacter sp. 638] | | |

TABLE 11b
acdH from Lactobacillus plantarum and proteins with with similar
amino acid sequence identity.

| | Identity | Accession |
|---|---|---|
| Description | (%) | number |
| acetaldehyde dehydrogenase [Lactobacillus | 100 | YP_004888365.1 |
| plantarum WCFS1] | | |
| acetaldehyde dehydrogenase [Lactobacillus | 95 | CCC16763.1 |
| pentosus IG1] | | |
| aldehyde-alcohol dehydrogenase | 58 | WP_016251441.1 |
| [Enterococcus cecorum] | | |
| aldehyde-alcohol dehydrogenase 2 | 57 | WP_016623694.1 |
| [Enterococcus faecalis] | | |
| bifunctional acetaldehyde-CoA/alcohol | 55 | WP_010493695.1 |
| dehydrogenase [Lactobacillus zeae] | | |
| alcohol dehydrogenase [Bacillus | 54 | WP_003280110.1 |
| thuringiensis] | | |
| bifunctional acetaldehyde-CoA/alcohol | 53 | WP_009931954.1 |
| dehydrogenase, partial [Listeria | | |
| monocytogenes] | | |

TABLE 11c
eutE from Escherichia coli and proteins with with similar amino
acid sequence identity.

| | Identity | Accession |
|---|---|---|
| Description | (%) | number |
| aldehyde oxidoreductase, ethanolamine | 100 | NP_416950.1 |
| utilization protein [Escherichia coli str. | | |
| K-12 substr. MG1655] | | |
| ethanolamine utilization; acetaldehyde | 99 | NP_289007.1 |
| dehydrogenase [Escherichia coli O157:H7 | | |
| str. EDL933] | | |
| aldehyde dehydrogenase [Escherichia | 99 | WP_001075674.1 |
| albertii] | | |

TABLE 11d
Lin1129 from Listeria innocua and proteins with with similar amino
acid sequence identity.

| | Identity | Accession |
|---|---|---|
| Description | (%) | number |
| aldehyde dehydrogenase [Listeria innocua] > | 100 | NP_470466.1 |
| emb\|CAC96360.1\| lin1129 [Listeria | | |
| innocua Clip11262] | | |
| ethanolamine utilization protein EutE | 99 | WP_003761764.1 |
| [Listeria innocua] | | |
| aldehyde dehydrogenase [Listeria | 95 | AGR09081.1 |
| monocytogenes] | | |
| hypothetical protein [Enterococcus | 64 | WP_010739890.1 |
| malodoratus] | | |
| aldehyde dehydrogenase [Yersinia aldovae] | 59 | WP_004699364.1 |
| aldehyde dehydrogenase EutE [Klebsiella | 58 | WP_004205473.1 |
| pneumoniae] | | |

TABLE 11e
adhE from Staphylococcus aureus and proteins with with similar amino
acid sequence identity.

| | Identity | Accession |
|---|---|---|
| Description | (%) | number |
| bifunctional acetaldehyde-CoA/alcohol | 100 | NP_370672.1 |
| dehydrogenase [Staphylococcus aureus | | |
| subsp. aureus Mu50] | | |
| aldehyde dehydrogenase family protein | 99 | YP_008127042.1 |
| [Staphylococcus aureus CA-347] | | |
| bifunctional acetaldehyde-CoA/alcohol | 85 | WP_002495347.1 |
| dehydrogenase [Staphylococcus | | |
| epidermidis] | | |
| aldehyde-alcohol dehydrogenase 2 | 75 | WP_016623694.1 |
| [Enterococcus faecalis] | | |

In an embodiment the recombinant yeast further comprises one or more nucleotide sequence encoding a acetyl-CoA synthetase (E.C. 6.2.1.1).

Acetyl-CoA synthetase (also known as acetate-CoA ligase and acetyl-activating enzyme) is a ubiquitous enzyme, found in both prokaryotes and eukaryotes, which catalyses the formation of acetyl-CoA from acetate, coenzyme A (CoA) and ATP as shown below:

ATP+acetate+CoA=AMP+diphosphate+acetyl-CoA

Preferably the endogenous ACS is overexpressed in the yeast cell.

Examples of suitable ACS are listed in table 12.

TABLE 12
ACS2 from Saccharomyces cerevisiae and proteins with similar
amino acid sequence.

| | Identity | Accession |
|---|---|---|
| Description | (%) | number |
| acetate-CoA ligase ACS2 [Saccharomyces | 100 | NP_013254.1 |
| cerevisiae S288c] | | |
| acetyl CoA synthetase [Saccharomyces | 99 | EDN59693.1 |
| cerevisiae YJM789] | | |
| acetate-CoA ligase [Kluyveromyces lactis | 85 | XP_453827.1 |
| NRRLY-1140] | | |
| acetate-CoA ligase [Candida glabrata | 83 | XP_445089.1 |
| CBS 138] | | |
| acetate-CoA ligase [Scheffersomyces | 68 | XP_001385819.15 |
| stipitis CBS 6054] | | |
| acetyl-coenzyme A synthetase FacA | 63 | EDP50475.1 |
| [Aspergillus fumigatus A1163] | | |
| acetate-CoA ligase facA-Penicillium | 62 | XP_002564696.110 |
| chrysogenum [Penicillium chrysogenum | | |
| Wisconsin 54-1255] | | |

In the process according to the invention and/or the recombinant yeast according to the invention any combination of the above mentioned protein(s) and/or nucleic acid sequences can be included.

Most preferably the recombinant yeast comprises a gene encoding for a protein comprising an amino acid sequence represented by SEQ ID NO: 6 or SEQ ID NO: 13 or a functional homologue of SEQ ID NO:6 having a sequence identity of at least 60%, at least 65%, at least 70%, at least 75%, at least 80%, at least 85%, at least 90%, at least 95%, at least 98% or at least 99% with SEQ ID No:6 or a functional homologue of SEQ ID NO:13 having a sequence identity of at least 60%, at least 65%, at least 70%, at least 75%, at least 80%, at least 85%, at least 90%, at least 95%, at least 98% or at least 99% with SEQ ID No:13.

Most Preferred Recombinant Yeast

As described above, most preferably the invention provides a recombinant yeast comprising:
a gene encoding for a heterologous protein having arabinose isomerase activity;
a gene encoding for a protein having ribulokinase activity;
a gene encoding for a protein having ribulose phosphate epimerase activity;
a gene encoding for a protein having acetaldehyde dehydrogenase activity, preferably acetylating acetaldehyde dehydrogenase activity;
a gene encoding for a protein having glycerol dehydrogenase activity;
a, preferably glucose tolerant, gene encoding for a protein having glycerol-proton symporter activity, preferably an STL1 protein;
a genetic modification causing overexpression of a nucleic acid sequence encoding for a homologous or heterologous protein having dihydroxyacetone kinase activity; and
a genetic modification causing deletion or disruption of a homologous protein with glycerol-efflux activity, preferably an fps1 protein native to the yeast.

It was advantageously found that the above specific combination of features does allow the recombinant yeast to quickly convert arabinose, even in the presence of glucose. In addition, surprisingly, the above combination is believed to also may allow for the co-conversion of arabinose and glucose, where some of the arabinose is already converted whilst the glucose is not depleted yet. This phenomenon is herein also referred to as simultaneous conversion, where some arabinose and some glucose are simultaneously converted.

Preferably the recombinant yeast further comprises, one, two or more copies of a heterologous gene encoding for one or more protein(s) having xylose isomerase activity; and/or one, two or more copies of a heterologous gene encoding for one or more protein(s) having xylose reductase activity; and/or one, two or more copies of a heterologous gene encoding for one or more protein(s) having xylitol dehydrogenase activity, as described in detail above.

In addition, the recombinant yeast preferably further comprises one or more genetic modifications for the overexpression of one or more enzymes of the (non-oxidative part) pentose phosphate pathway, as described in detail above.

Further preferences are as described for the recombinant yeast in the description above.

For example, the recombinant yeast may suitably be a recombinant yeast comprising:
a gene encoding for a heterologous protein comprising an amino acid sequence represented by SEQ ID NO: 10 or a functional homologue of SEQ ID NO:10 having a sequence identity of at least 60%, at least 65%, at least 70%, at least 75%, at least 80%, at least 85%, at least 90%, at least 95%, at least 98% or at least 99% with SEQ ID No:10;
a gene encoding for a protein comprising an amino acid sequence represented by SEQ ID NO: 11 or a functional homologue of SEQ ID NO:11 having a sequence identity of at least 60%, at least 65%, at least 70%, at least 75%, at least 80%, at least 85%, at least 90%, at least 95%, at least 98% or at least 99% with SEQ ID No:11;
a gene encoding for a protein comprising an amino acid sequence represented by SEQ ID NO: 12 or a functional homologue of SEQ ID NO:12 having a sequence identity of at least 60%, at least 65%, at least 70%, at least 75%, at least 80%, at least 85%, at least 90%, at least 95%, at least 98% or at least 99% with SEQ ID No:12;
a gene encoding for a protein comprising an amino acid sequence represented by SEQ ID NO: 6 or SEQ ID NO: 13 or a functional homologue of SEQ ID NO:6 having a sequence identity of at least 60%, at least 65%, at least 70%, at least 75%, at least 80%, at least 85%, at least 90%, at least 95%, at least 98% or at least 99% with SEQ ID No:6 or a functional homologue of SEQ ID NO: 13 having a sequence identity of at least 60%, at least 65%, at least 70%, at least 75%, at least 80%, at least 85%, at least 90%, at least 95%, at least 98% or at least 99% with SEQ ID No: 13;
a gene encoding for a protein comprising an amino acid sequence represented by SEQ ID NO: 7 or a functional homologue of SEQ ID NO:7 having a sequence identity of at least 60%, at least 65%, at least 70%, at least 75%, at least 80%, at least 85%, at least 90%, at least 95%, at least 98% or at least 99% with SEQ ID No:7;
a gene encoding for a protein comprising an amino acid sequence represented by SEQ ID NO: 1, 2, 3, 4 or 5 or a functional homologue of respectively SEQ ID NO:1, 2, 3, 4 or 5 having a sequence identity of at least 60%, at least 65%, at least 70%, at least 75%, at least 80%, at least 85%, at least 90%, at least 95%, at least 98% or at least 99% with respectively SEQ ID No:1, 2, 3, 4 or 5;
a genetic modification causing overexpression of a nucleic acid sequence encoding for a protein comprising an amino acid sequence represented by SEQ ID NO: 8 or SEQ ID NO: 9 or a functional homologue of SEQ ID NO:8 having a sequence identity of at least 60%, at least 65%, at least 70%, at least 75%, at least 80%, at least 85%, at least 90%, at least 95%, at least 98% or at least 99% with SEQ ID No:8 or a functional homologue of SEQ ID NO:9 having a sequence identity of at least 60%, at least 65%, at least 70%, at least 75%, at least 80%, at least 85%, at least 90%, at least 95%, at least 98% or at least 99% with SEQ ID No:9;
a genetic modification leading to the deletion or disruption of a homologous protein comprising an amino acid sequence represented by SEQ ID NO: 14 or a functional homologue of SEQ ID NO:14 having a sequence identity of at least 60%, at least 65%, at least 70%, at least 75%, at least 80%, at least 85%, at least 90%, at least 95%, at least 98% or at least 99% with SEQ ID No:14.

Again, further preferences are as described for the recombinant yeast in the description above.

Fermentation

In the process according to the invention and/or the recombinant yeast according to the invention any combination of the above mentioned protein(s) and/or nucleic acid sequences can be included and preferences mentioned above for the recombinant yeast can be applied for both the process according to the invention as well as the recombinant yeast according to the invention.

The process according to the invention comprises fermenting of a carbon source composition with a recombinant yeast. As indicated above, the carbon source composition may comprise, in addition to glucose and arabinose, non-sugar compounds such as glycerol and/or acetic acid or a salt thereof (acetate).

If the carbon source composition comprises glucose, arabinose and glycerol, preferably each of such glucose, arabinose and glycerol is converted into ethanol.

If the carbon source composition comprises glucose, arabinose and acetic acid (or a salt thereof), preferably each of such glucose, arabinose and acetic acid (or a salt thereof) is converted into ethanol.

If the carbon source composition comprises glucose, arabinose, glycerol and acetic acid (or a salt thereof), preferably each of such glucose, arabinose, glycerol and acetic acid (or a salt thereof) is converted into ethanol.

Hence, the present invention further provides a process as described above, wherein further the carbon source composition comprises at least glucose, arabinose and glycerol; and wherein further the recombinant yeast comprises arabinose isomerase activity, ribulokinase activity and ribulose phosphate epimerase activity, glycerol dehydrogenase activity, dihydroxyacetone kinase activity, and glycerol-proton symporter activity; and wherein the recombinant yeast further comprises a genetic modification leading to the reduction, inhibition or elimination of the activity of one or more homologous protein(s) with glycerol-efflux activity; and wherein each of the glucose, the arabinose and the glycerol is converted into ethanol.

Also, the present invention provides a process as described above, wherein further the carbon composition further comprises acetic acid, or a salt thereof; and wherein the recombinant yeast further comprises one or more nucleic acid sequence(s) encoding for one or more protein(s) having heterologous acetylating acetaldehyde dehydrogenase activity; and wherein the acetic acid, or the salt thereof, is converted to ethanol.

And in addition, the present invention provides a process as described above, wherein the carbon composition further comprises acetic acid, or a salt thereof; and wherein the recombinant yeast further comprises one or more nucleic acid sequence(s) encoding for one or more protein(s) having heterologous acetylating acetaldehyde dehydrogenase activity; and wherein the acetic acid, or the salt thereof, is converted to ethanol.

The recombinant yeast advantageously allows for the anaerobic simultaneous arabinose and glucose consumption. Further the invention relates to a process wherein advantageously the fermentation time for substantially complete fermentation of glucose and/or arabinose and/or glycerol and/or acetic acid (or a salt thereof) is reduced relative to the corresponding fermentation of wild-type yeast. In a preferred process the fermentation time for arabinose is reduced by 20% or more, preferably 40% or more. In a preferred process, pentose and glucose are co-fermented. In a preferred process the overall ethanol production rate is at least about 10%, at least about 20%, at least about 50% or about 100% higher than that of a process with the corresponding wild-type yeast.

In a preferred process the carbon source composition comprises a hydrolysate of lignocellulosic material. The hydrolysate may be an enzymatic hydrolysate of lignocellulosic material.

The fermentation is preferably carried out, preferably in a suitable fermentation reactor, preferably under anaerobic conditions, preferably in the absence of oxygen or in which substantially no oxygen is consumed, preferably less than about 5, about 2.5 or about 1 mmol/L/h, more preferably 0 mmol/L/h is consumed (i.e. oxygen consumption is not detectable), and wherein organic molecules serve as both electron donor and electron acceptors.

The fermentation process is preferably run at a temperature that is optimal for the recombinant yeast. Preferably the fermentation is carried out at a temperature which is less than about 42° C., preferably less than about 38° C. Preferably the fermentation is carried out at a temperature which is lower than about 35° C., about 33° C., about 30° C. or about 28° C. and at a temperature which is higher than about 20° C., about 22° C., or about 25° C.

The ethanol yield, suitably calculated on carbon base, is preferably at least 50 wt %, more preferably at least 60 wt %, still more preferably at least 70 wt %, even more preferably at least 75 wt %, still even more preferably at least 80 wt %, yet even more preferably at least 85 wt % and most preferably at least 90 wt %, based on carbon sources contained in the carbon source composition. By a calculation on carbon base is herewith understood: the weight of carbon molecules in the ethanol product, divided by the combined weight of carbon molecules in the carbon sources in the carbon source composition.

The process may be carried out in batch, fed-batch or continuous mode. A separate hydrolysis and fermentation (SHF) process or a simultaneous saccharification and fermentation (SSF) process may also be applied. A combination of these fermentation process modes may also be possible for optimal productivity.

For the recovery of the ethanol and other products such as DDGS existing technologies can be used. Suitable methods for recovering ethanol from the fermentation mixture include fractionation and adsorption techniques. For example, a beer still can be used to process the fermented product, which contains ethanol in an aqueous mixture, to produce an enriched ethanol-containing mixture that is then subjected to fractionation (e.g., fractional distillation or other like techniques). Next, the fractions containing the highest concentrations of ethanol can be passed through an adsorber to remove most, if not all, of the remaining water from the ethanol.

Hence, as explained above, most preferably the invention provides a process for production of ethanol, such process comprising fermenting of a carbon source comprising at least glucose, arabinose, glycerol and acetic acid (or a salt thereof), with a recombinant yeast as described above, wherein each of the glucose, arabinose, glycerol and acetic acid are converted into ethanol and wherein most preferably glucose and arabinose are converted into ethanol simultaneously. Such simultaneous conversion is herein understood to refer to a situation where at least some of the arabinose is being converted whilst at the same time also some glucose is being converted. Even more preferably, the carbon source further comprises xylose, which xylose is also converted into ethanol.

The following prophetic examples are non-limiting and intended to be purely illustrative.

### EXAMPLES

### Materials and Methods

General Molecular Biology Techniques

Unless indicated otherwise, the methods used are standard biochemical techniques. Examples of suitable general methodology textbooks include Sambrook et al., Molecular Cloning, a Laboratory Manual (1989) and Ausubel et al., Current Protocols in Molecular Biology (1995), John Wiley & Sons, Inc.

Media

The media used in the experiments can for example be YEP-medium (10 g/l yeast extract, 20 g/l peptone) or solid YNB-medium (6.7 g/l yeast nitrogen base, 15 g/l agar), supplemented with sugars as indicated in the examples. For solid YEP medium, 15 g/l agar can be added to the liquid medium prior to sterilization.

In the anaerobic screening experiment, Mineral Medium can be used. The composition of Mineral Medium has been described by Verduyn et al. (Yeast (1992), Volume 8, 501-517). The use of ammoniumsulfate can however be omitted; instead, as a nitrogen source, 2.3 g/l urea can be used. In addition, ergosterol (0.01 g/L), Tween80 (0.42 g/L) and sugars (as indicated) can be added.

Strains

The strain used as a reference in the below prophetic examples comprises arabinose isomerase activity, ribulokinase activity, ribulose phosphate epimerase activity and glycerol-proton symporter activity and further xylulose kinase activity, NAD+-dependent glycerol dehydrogenase activity, dihydroxyacetone kinase activity and acetylating acetaldehyde dehydrogenase activity. Such strains, their construction and their genotype, are for example described in WO 2015/028583 in the form of the transformants T1, T2, T3, T4 and T5 in table 11 thereof. For the below prophetic example reference is made to transformant T5 as listed in table 11 of WO 2015/028583, further herein below referred to as simply “T5”.

Strain Construction

The strain construction approach is described in patent application PCT/EP2013/056623. It describes the techniques enabling the construction of expression cassettes from various genes of interest in such a way, that these cassettes are combined into a pathway and integrated in a specific locus of the yeast genome upon transformation of this yeast.

Firstly, an integration site in the yeast genome can be chosen (e.g. INT1). A DNA fragment of approximately 500 bp of the up- and downstream part of the integration locus can be amplified using PCR, flanked by a connector. These connectors are 50 bp sequences that allow for correct in vivo recombination of the pathway upon transformation in yeast (Saccharomyces cerevisiae e.g.). The genes of interest, as well as a selectable resistance marker (e.g. kanMX, natMX or an equivalent), can be generated by PCR, incorporating a different connector at each flank. Upon transformation of yeast cells with the DNA fragments, in vivo recombination and integration into the genome can take place at the desired location. This technique allows for pathway tuning, as one or more genes from the pathway can be replaced with (an)other gene(s) or genetic element(s), as long as that the connectors that allow for homologous recombination remain constant (patent application PCT/EP2013/056623).

Expression Cassette Construction

The open reading frames (ORFs), promoter sequences and terminators can be synthesized at for example DNA 2.0 (Menlo Park, Calif. 94025, USA). The promoter, ORF and terminator sequences can be recombined by using the Golden Gate technology, as described by Engler et al (2011) and references therein. A plasmid containing a glycerol transporter expression cassettes can be used as included therein as SEQ ID NO: 36.

Expression Cassette Amplification and Transformation of the Yeast Cells

The expression cassettes can be amplified by PCR using suitable primers

Yeast transformation can be done according to the method described by Schiestl and Gietz (Current Genetics (1989), Volume 16, 339-346).

Anaerobic Growth Experiments in Microplates

Growth experiments can be performed in flat bottom NUNC microplates (MTPs). 275 μl of medium can be filled out in each well.

The composition of the medium can be similar to that described in WO2015/028583, further including arabinose, as follows: 20 g/l glucose; 20 g/l xylose; 20 g/l arabinose; 10 g/I glycerol; 2 g/l acetic acid. Initial pH can be pH 4.5. All MTPs can be sealed with an aluminum seal. The MTPs can be then placed in the anaerobic incubator (Infors). After 48 hours of growth, the MTPs can be removed from the anaerobic Infors. The cells can then be spun down by centrifuging 10 minutes@2750 rpm in a microplate centrifuge. 150 μl of the supernatant can then be transferred to a MTP suitable for NMR analysis.

AFM Experiments

The Alcohol Fermentation Monitor (AFM; Halotec, Veenendaal, the Netherlands) is a robust and user-friendly laboratory parallel bioreactor that allows for accurate comparisons of carbon conversion rates and yields for six simultaneous anaerobic fermentations. The starting culture of the AFM experiment can contain 50 mg of yeast (dry weight). To determine this, a calibration curve can be made of the RN1041 strain of biomass vs. OD700. This calibration curve can be used in the experiment to determine the volume of cell culture needed for 50 mg of yeast (dry weight).

Prior to the start of the AFM experiment, pre-cultures can be grown as suitable. For each strain the OD700 can be measured and 50 mg of yeast (dry weight) can be inoculated in 400 ml Mineral Medium (Verduyn et al. (Yeast (1992), Volume 8, 501-517), supplemented with 2.3 g/I urea (instead of ammonium sulfate) and carbon sources as indicated in the examples.

NMR Analysis

For the quantification of glucose, xylose, arabinose, glycerol, acetic acid and ethanol in a sample, 1D ¹H NMR spectra can be recorded on a Bruker Avance III 700 MHz, equipped with a cryo-probe, using a pulse program with water suppression (power corresponding to 3 Hz) at a temperature of 27° C.

### Prophetic Comparative Example A

Fermentation Characteristics of Strain T5

To generate a pre-culture, a strain like T5 can be pre-grown under aerobic conditions in Mineral Medium supplemented with 2.3 grams urea per liter, 20 g/l glucose; 20 g/l xylose; 20 g/I arabinose and initial pH 4.5. Incubation can be overnight at 30° C. and 280 rpm. The following day, the optical density at 600 nm can be determined and cells can be spun down by centrifugation.

Subsequently, 400 ml of Mineral Medium containing per liter approximately 20 grams of glucose, 20 grams of xylose, 20 grams of arabinose, 10 grams of glycerol and 2 grams of acetic acid can be inoculated with transformant strain T5 in order to perform an AFM experiment. After 48 hours, cells can be separated from the supernatant and the supernatant can be analyzed by NMR.

Prophetic results of this experiment are given in Table 13 below.

TABLE 13
Prophetic results for comparative example A, representing average
medium composition before (Medium) and after incubation with
transformant strain T5 for 48 hours.

| Component | g/l at time = 0 hours | g/l at time = 48 hours |
|---|---|---|
| glucose | 20 | 0.2 |
| xylose | 20 | 0.3 |
| arabinose | 20 | 9 |
| glycerol | 10 | 7.6 |
| acetic acid | 2 | 1.2 |
| ethanol | 0 | 25 |

Transformant strain T5 may result in a commercially interesting yield, but ethanol yields would even be higher if arabinose could be converted more and/or quicker.

### Prophetic Example 1

Construction of FPS1 Knock-Out Transformant Strain T5-fps1Δ

In order to experimentally verify the effect of reduction, inhibition or elimination of the activity of one or more homologous protein(s) with glycerol-efflux activity, the FPS1 gene in the above mentioned Transformant strain T5 can be deleted, for example via the homologous recombination technique as exemplified by Kyung Ok Yu et al, in their article titled “Reduction of glycerol production to improve ethanol yield in an engineered Saccharomyces cerevisiae using glycerol as a substrate”, published in the Journal of Biotechnology vol. 150 (2010), pages 209-214. The FPS1 deletion strains are herein referred to as strain T5-fps1Δ

Fermentation Characteristics of Strain T5-fps1Δ

To generate a pre-culture, knock-out strain T5-fps1Δ can be pre-grown under aerobic conditions in Mineral Medium supplemented with 2.3 grams urea per liter, 20 g/l glucose; 20 g/I xylose; 20 g/l arabinose and initial pH 4.5. Incubation can be overnight at 30° C. and 280 rpm. The following day, the optical density at 600 nm can be determined and cells can be spun down by centrifugation.

Subsequently, 400 ml of Mineral Medium containing per liter approximately 20 grams of glucose, 20 grams of xylose, 20 grams of arabinose, 10 grams of glycerol and 2 grams of acetic acid can be inoculated with knock-out strain T5-fps1Δ in order to perform an AFM experiment. After 48 hours, cells can be separated from the supernatant and the supernatant can be analyzed by NMR.

Prophetic results of this experiment are given in Table 14 below.

TABLE 14
Prophetic results for example 1, representing average medium
composition before (Medium) and after incubation with T5-fps1Δ
for 48 hours.

| Component | g/l at time = 0 hours | g/l at time = 48 hours |
|---|---|---|
| glucose | 20 | 0.2 |
| xylose | 20 | 0.3 |
| arabinose | 20 | 4.0 |
| glycerol | 10 | 0.4 |
| acetic acid | 2 | 0.4 |
| ethanol | 0 | 33 |

It is illustrated that a substantial amount of the carbon supplied in the feed as glucose, xylose, arabinose, glycerol and acetic acid can advantageously be converted into ethanol.

### Example 2

In order to experimentally verify the above prophetic findings, an actual yeast strain was constructed comprising arabinose isomerase activity, ribulokinase activity, ribulose phosphate epimerase activity and glycerol-proton symporter activity and further xylulose isomerase activity, NAD+-dependent glycerol dehydrogenase activity, dihydroxyacetone kinase activity and acetylating acetaldehyde dehydrogenase activity. In addition the FPS1 gene was knocked out.

The yeast strain was constructed by applying a number of genetic modifications in a Saccharomyces cerevisiae host cell. This yeast strain is herein below termed YS1.

As a basis for the YS1 strain a S. cerevisiae strain similar to the strains as described in WO2011/003893 and WO2011/131667 was used. That is, this basis strain comprised multiple copies of the araA, araB and araD genes and multiple copies of xylose isomerase gene (xylA). In addition, this basis strain comprised a deletion of one of the gpd1 genes, where such gpd1 gene was replaced by synthetic DNA, codon optimized for expression in Saccharomyces cerevisiae, encoding for the ethanolamine utilizing protein, an acetylating acetaldehyde dehydrogenase (eutE) from Escherichia coli. A second gpd1 gene remained present.

The basis strain was subsequently genetically modified using CRISPR-Cas9 technology (as described for yeast in the article by DiCarlo et al., titled “Genome engineering in Saccharomyces cerevisiae using CRISPR-Cas systems”, published in 2013, Nucleic Acids Res 41:4336-4343), nowadays well known by a person skilled in the art. Cas9-expressing plasmids and integration site-specific gRNA-expressing plasmids were introduced to the basis strain.

Four expression cassettes for expression in S. cerevisiae were generated on plasmids in the intermediate host E. coli, by cloning the coding sequences between a S. cerevisiae-derived promoter and a S. cerevisiae-derived terminator. After plasmid DNA isolation, the expression cassettes were cut or PCR-amplified from the plasmids and transformed into the S. cerevisiae basis strain.

The cassettes included a first cassette for integration of synthetic DNA, codon optimized for expression in Saccharomyces cerevisiae, encoding for the glycerol transporter from Zygosaccharomyces rouxii, suitably having a protein sequence as listed in SEQ ID NO: 5. However, as described above also the glycerol transporters having protein sequences as listed in SEQ ID NO's 1 to 4 can be used.

The cassettes included also a second cassette for integration of synthetic DNA, codon optimized for expression in Saccharomyces cerevisiae, encoding for the glycerol dehydrogenase of Escherichia coli (gldA), suitably having a protein sequence as listed in SEQ ID NO: 7. However, as described above also other glycerol dehydrogenases as mentioned herein can be used.

The cassettes further included a third cassette for integration of synthetic DNA, codon optimized for expression in Saccharomyces cerevisiae, encoding for nucleic acid sequences allowing overexpression of the dihydroxyacetone kinase 1 (DAK1) of Saccharomyces cerevisiae, suitably having a protein sequence as listed in SEQ ID NO: 8. However, as described above also other dihydroxyacetone kinase as mentioned herein can be used.

In addition, the cassettes included a fourth cassette for integration of synthetic DNA, codon optimized for expression in Saccharomyces cerevisiae, encoding for the ethanolamine utilizing protein, an acetylating acetaldehyde dehydrogenase (Ec_eutE) from Escherichia coli, suitably having a protein sequence as listed in SEQ ID NO: 13. However, as shown herein above, also alternatives such as the enzyme with the protein sequence listed as SEQ ID NO: 6 can be used.

The four cassettes were placed at the position of the fps1 gene of the basis strain, hence knocking out the FPS1 protein. The resultant strain was termed YS1.

Growth Experiment in Microplates

The above strain YS1 was used in MTP growth experiments performed in Deepwell plates 96×1.2 mL (VWR) (MTPs), with 350 μl of medium.

First a pre-culturing step was performed in which in the medium consisted of 90% YEPh-D (10 g/L yeast extract+20 g/L BBL Phytone Peptone, 20 g/L glucose) and 10% separated corn fiber hydrolysate. The composition of the corn fiber hydrolysate with regards to the relevant components was as follows: ˜60 g/l glucose; 25 g/l xylose; 20 g/l arabinose; 0 g/l glycerol; 2-3 g/I acetic acid. Before use the solids were removed by centrifugation and pH was set to pH 5.5 using ammonia. The MTPs were inoculated using biomass scraped from agar. Plates were sealed and incubated for 2 days at 32° C., 750 rpm at 80% humidity in an incubator shaker (INFORS HT).

The main fermentation was subsequently performed in a medium consisting of separated corn fiber hydrolysate supplemented with 50 g/L glucose. The composition of the corn fiber hydrolysate was as indicated above and with regards to the relevant components comprised: ˜60 g/I glucose; 25 g/l xylose; 20 g/l arabinose; 0 g/l glycerol; 2-3 g/l acetic acid. To such corn fiber hydrolysate, 50 g/L of additional glucose was added.

The MTP plates were sealed to create anaerobic conditions and cultures were grown at 32° C., 250 rpm and 80% humidity in an incubator shaker (INFORS HT). Sampling was performed at 20 h, 26 h or 48 h. Sampling was performed as follows: the MTPs were spun down by centrifuging 10 minutes @ 2750 rpm in a microplate centrifuge. In this example 2, 100 μl of the supernatant was taken from the MTP plates and was mixed with 100 μl internal NMR standard solution and diluted with 500 μl D₂O as described in detail below under the analysis section.

The achieved conversions of glucose, arabinose, xylose, glycerol and acetic acid are illustrated below for YS1 in table 15.

Analysis of Glucose, Xylose, Arabinose, Glycerol, Acetic Acid and Ethanol

For the quantification of glucose, xylose, arabinose, glycerol, acetic acid and ethanol, 100 μl of the supernatant sample was transferred accurately into a suitable vial. Subsequently 100 μl internal standard solution, (maleic acid (20 g/l, EDTA 40 g/l, 4,4-dimethyl-4-silapentane-1-sulfonic acid 0.5 g/L, and sodium hydroxide set to pH 6.40 in D₂O) was added to the sample. This mixture was further diluted with 500 μl D₂O.

In contrast to the NMR method as described herein above, for this example 2 a different NMR spectrometer was used, namely a Bruker Avance III spectrometer, operating at a proton frequency of 400 MHz. 1D ¹H NMR spectra of the clear solution were recorded on this Bruker Avance III spectrometer, operating at a proton frequency of 400 MHz, equipped with a prodigy probe, using a pulse program with water suppression (ZGCPPR, solvent suppression power of 5 Hz) at a temperature of 300 K, a 90 degree excitation pulse was applied followed by acquisition time of 2.0 seconds and a relaxation delay of 1.2 seconds. The number of scans was set at 8, preceded by 4 dummy scans.

The analyte concentrations (in gram per liter) were calculated with the below formula, based on the below signals (δ relative to DSS):

Pₓ=(Aₓ/Aₛₜ)×(nₛₜ/nₓ)×(Mwₓ/Mwₛₜ)×(Mₜ/Wₓ)×Pₛₜ

wherein:
x=compound to be determined
st=standard
n=number of protons of the signal in question
Mw=molecular weight
W=amount weighted
P=purity
A=area

Signals:
Glucose: second peak from β-H1 glucose signal (d, 4.63 ppm, 0.643H, J=8 Hz) at 4.62 ppm, n=0.3215 H
Xylose: second peak from β-H1 (d, 4.56 ppm, 0.647H, J=8 Hz) at 4.56 ppm, n=0.3235H.
Arabinose: α-H1 (d, 4.50 ppm, 0.622H, J=8 Hz)
Gycerol: first peak from H1/H3 signals (dd, 3.55 ppm, 2H, J=7 Hz, 12 Hz), at 3.55 ppm, n=0.7H
Acetic acid: (s, 1.90 ppm, 3H)
Ethanol: (t, 1.17 ppm, 3H, J=7 Hz)
   As signal for the internal standard, maleic acid was used having a peak around 6.10 ppm (S, 2H)

TABLE 15
Results for example 2, representing average medium composition
during fermentation with YS1 at a time of 0, 20, 26 and 48 hours.

| | g/l at time = | g/l at time = | g/l at time = | g/l at time = |
|---|---|---|---|---|
| Component | 0 hours | 20 hours | 26 hours | 48 hours |
| glucose | 109.8 ± 0.9 | 10.6 ± 1.7 | 0 | 0 |
| xylose | 24.6 ± 0.3 | 24.0 ± 0.4 | 20.6 ± 0.6 | 2.9 ± 2.6 |
| arabinose | 20.1 ± 0.1 | 19.8 ± 0.2 | 18.5 ± 0.3 | 6.4 ± 0.4 |
| glycerol | 0 | 0 | 0 | 0 |
| acetic acid | 2.88 ± 0.04 | 2.23 ± 0.06 | 2.2 ± 0.08 | 0 |
| ethanol | — | 29.9 ± 0.4 | 34.0 ± 0.4 | 41.3 ± 0.6 |

As illustrated by the results in Table 15, glucose repression could advantageously be avoided. Advantageously substantial amounts of each of glucose, xylose, arabinose, glycerol and acetic acid were converted into ethanol.

In respect of the glycerol it is noted that in line with normal fermentations, glycerol is believed to be formed during the fermentation. However, as glycerol is quickly consumed again, the amount of glycerol never reaches the detection limit.

In addition, the data suggests that advantageously and remarkably, at a time of 20 hours of fermentation, the conversion of some of the arabinose and/or xylose may already have started whilst the glucose has not been completely converted yet. This suggests that the specific combination of features in the claimed recombinant yeast and process according to the invention may advantageously allow for co-conversion of pentoses such as xylose and/or arabinose simultaneously with the conversion of a hexose such as glucose.

1. A process for production of ethanol, the process comprising
fermenting a carbon source composition with a recombinant yeast,
wherein the carbon source composition comprises at least glucose and arabinose; and
wherein the recombinant yeast comprises arabinose isomerase activity, ribulokinase activity, ribulose phosphate epimerase activity, glycerol uptake activity and glycerol conversion capacity; and
wherein the recombinant yeast further comprises a genetic modification leading to reduction, downregulation, inhibition and/or elimination of activity of a homologous protein with glycerol-efflux activity; and
wherein each of the glucose and the arabinose is converted into ethanol.

2. The process according to claim 1, wherein the recombinant yeast comprises a bacterial gene encoding a heterologous protein with arabinose isomerase activity; a bacterial gene encoding a heterologous protein with ribulokinase activity; and a bacterial gene encoding a heterologous protein with ribulose phosphate epimerase activity.

3. The process according to claim 1, wherein the recombinant yeast comprises a glucose-tolerant gene encoding a heterologous protein with glycerol-proton symporter activity.

4. The process according to claim 1, wherein the recombinant yeast comprises one or more glucose-tolerant STL genes.

5. The process according to claim 1, wherein the recombinant yeast comprises, one, two or more copies of a heterologous gene encoding for one or more protein(s) having xylose isomerase activity; and/or one, two or more copies of a heterologous gene encoding for one or more protein(s) having xylose reductase activity; and/or one, two or more copies of a heterologous gene encoding for one or more protein(s) having xylitol dehydrogenase activity.

6. The process according to claim 1, wherein activity of one or more homologous aquaglyceroporines with glycerol-efflux activity in the recombinant yeast is reduced, inhibited or eliminated.

7. The process according to claim 1, wherein activity of homologous fps1 in the recombinant yeast is reduced, inhibited or eliminated.

8. The process according to claim 1, wherein a homologous FPS1 gene of the recombinant yeast is disrupted or knocked out.

9. The process according to claim 1,
wherein the carbon source composition comprises at least glucose, arabinose and glycerol; and
wherein the recombinant yeast comprises arabinose isomerase activity, ribulokinase activity and ribulose phosphate epimerase activity, glycerol dehydrogenase activity, dihydroxyacetone kinase activity, and glycerol-proton symporter activity; and
wherein the recombinant yeast further comprises a genetic modification leading to reduction, inhibition or elimination of activity of one or more homologous protein(s) with glycerol-efflux activity; and
wherein each of the glucose, the arabinose and the glycerol is converted into ethanol.

10. The process according to claim 9, wherein the recombinant yeast comprises a nucleic acid sequence encoding an enzyme with NAD+-dependent glycerol dehydrogenase activity.

11. The process according to claim 9, wherein the recombinant yeast comprises a bacterial gene encoding an enzyme with NAD+-dependent glycerol dehydrogenase activity.

12. The process according to claim 1,
wherein the carbon source composition further comprises acetic acid, or a salt thereof; and
wherein the recombinant yeast further comprises
a gene encoding for a heterologous protein having acetyl-Coenzyme A synthetase activity; and/or
a gene encoding for a heterologous protein having acetylating acetaldehyde dehydrogenase activity; and
wherein the acetic acid, or the salt thereof, is converted to ethanol.

13. The process according to claim 1, wherein at least part of the fermenting is carried out under anaerobic conditions.

14. A recombinant yeast comprising:
a gene encoding a heterologous protein having arabinose isomerase activity;
a gene encoding a heterologous protein having ribulokinase activity;
a gene encoding a heterologous protein having ribulose phosphate epimerase activity;
a gene encoding a heterologous protein having glycerol uptake activity;
a gene encoding a heterologous protein having NAD+-dependent glycerol dehydrogenase activity; and
a genetic modification leading to the reduction, downregulation, inhibition and/or elimination of the activity of a homologous protein with glycerol-efflux activity.

15. The recombinant yeast according to claim 14, further comprising:
one or more nucleic acid sequence(s) encoding for a heterologous NADH-oxidizing enzyme or enzymatic pathway.

16. The recombinant yeast according to claim 14 or 15, further comprising:
a gene encoding a heterologous enzyme having acetyl-Coenzyme A synthetase activity; and/or
a gene encoding a heterologous enzyme having, optionally, acetylating, acetaldehyde dehydrogenase activity.


SEQUENCE LISTING (14 sequences)

SEQ ID NO: 1 - protein, 598 aa, Schizosaccharomyces pombe
  MISC_FEATURE (1)..(598): Amino acid sequence of the glycerol transporter from S. pombe
MSVPLRFSTPSSSPSASDNESVHDDGPTTELDTFNTTDVPRRVNTTKARQMRPKNTLKVAFSSPNLKGLDNTADSDSQPWLGGYLAGRLEDISGQSRRNYVDPYYEELNAGRRPNKPVWSLNGPLPHVLGNSVVEKISQKNQEARSRANSRVNSRANSRANSSVSLAGMDGSPNWKRKMKSAVFGSRVKLNDEEAQLPRNKSSVSIAEQAASRPKVSFSLQSSRQPSIAEEQPQTQRKSSAITVEHAENAEPETPRNNVSFSRKPSIAEQDSSQDITMPPNEIIAEESLDSGSDTETLYLNYWCKIRHFFREGFAEFLGTLVLVVFGVGSNLQATVTNGAGGSFESLSFAWGFGCMLGVYIAGGISGGHVNPAVTISLAIFRKFPWYKVPIYIFFQIWGAFFGGALAYGYHWSSITEFEGGKDIRTPATGGCLYTNPKPYVTWRNAFFDEFIGTAVLVGCLFAILDDTNSPPTQGMTAFIVGLLIAAIGMALGYQTSFTLNPARDLGPRMFAWWIGYGPHSFHLYHWWWTWGAWGGTIGGGIAGGLIYDLVIFTGPESPLNYPDNGFIDKKVHQITAKFEKEEEVENLEKTDSPIENN

SEQ ID NO: 2 - protein, 258 aa, Plasmodium falciparum
  MISC_FEATURE (1)..(258): Amino acid sequence of the glycerol transporter from Plasmodium falciparum
MHMLFYKSYVREFIGEFLGTFVLMFLGEGATANFHTTGLSGDWYKLCLGWGLAVFFGILVSAKLSGAHLNLAVSIGLSSINKFDLKKIPVYFFAQLLGAFVGTSTVYGLYHGFISNSKIPQFAWETSRNPSISLTGAFFNELILTGILLLVILVVVDENICGKFHILKLSSVVGLIILCIGITFGGNTGFALNPSRDLGSRFLSLIAYGKDTFTKDNFYFWVPLVAPCVGSVVFCQFYDKVICPLVDLANNEKDGVDL

SEQ ID NO: 3 - protein, 291 aa, Danio rerio
  MISC_FEATURE (1)..(291): Amino acid sequence of the glycerol transporter from Danio rerio
MEYLENIRNLRGRCVLRRDIIREFLAELLGTFVLILFGCGSVAQTVLSREAKGQLLTIHFGFTLGVMLAVYMAGGVSGGHVNPAVSLAMVVLRKLPLKKFPVYVLAQFLGAFFGSCAVYCLYYDAFTEFANGELAVTGPNVTAGIFASYPREGLSLLNGFIDQVIGAGALVLCILAVVDKKNIGAPKGMEPLLVGLSILAIGVSMALNCGYPINPARDLGPRLFTAIAGWGLTVFSAGNGWWWVPVVGPMVGGVVGAAIYFLMIEMHHPENDKNLEDDNSLKDKYELNTVN

SEQ ID NO: 4 - protein, 292 aa, Xenopus (Silurana) tropicalis
  MISC_FEATURE (1)..(292): Amino acid sequence of the glycerol transporter from Xenopus tropicalis
MGRQKDFVNKCNQMLRLRNKLLRQALSECLGTLILVMFGCGSVAQVVLSKGSHGQFLTVNLAFGFAVMLGILISGQVSGGHLNPAVTFALCILAREPWVKFPVYSIAQTLGAFLGAGIIYGLYYDAIWYFANDQLYVTGENGTAGIFTTFPSDHLTLMNGFFDQFIGTAALVVCVLAIVDPNNNPIPRGLEAFTVGFVVLVIGTSMGFNSGYAVNPARDFGPRLFTSLAGWGTEVFWAGNQWWWVPIVSPLLGAFAGVLVYQLMIGCHIEPVPQSTEQENIKLADVKPKDRI

SEQ ID NO: 5 - protein, 592 aa, Zygosaccharomyces rouxii
  MISC_FEATURE (1)..(592): Amino acid sequence of the glycerol transporter from Z. rouxii
MGKRTQGFMDYVFSRTSTAGLKGARLRYTAAAVAVIGFALFGYDQGLMSGLITGDQFNKEFPPTKSNGDNDRYASVIQGAVTACYEIGCFFGSLFVLFFGDAIGRKPLIIFGAIIVIIGTVISTAPFHHAWGLGQFVVGRVITGVGTGFNTSTIPVWQSEMTKPNIRGAMINLDGSVIAFGTMIAYWLDFGFSFINSSVQWRFPVSVQIIFALVLLFGIVRMPESPRWLMAKKRPAEARYVLACLNDLPENDDAILAEMTSLHEAVNRSSNQKSQMKSLFSMGKQQNFSRALIASSTQFFQQFTGCNAAIYYSTVLFQTTVQLDRLLAMILGGVFATVYTLSTLPSFYLVEKVGRRKMFFFGALGQGISFIITFACLVNPTKQNAKGAAVGLYLFIICFGLAILELPWIYPPEIASMRVRAATNAMSTCTNWVTNFAVVMFTPVFIQTSQWGCYLFFAVMNFIYLPVIFFFYPETAGRSLEEIDIIFAKAHVDGTLPWMVAHRLPKLSMTEVEDYSQSLGLHDDENEKEEYDEKEAEANAALFQVETSSKSPSSNRKDDDAPIEHNEVQESNDNSSNSSNVEAPIPVHHNDP

SEQ ID NO: 6 - protein, 891 aa, Escherichia coli
  MISC_FEATURE (1)..(891): Amino acid sequence of acetylating acetaldehyde dehydrogenase from Escherichia coli
MAVTNVAELNALVERVKKAQREYASFTQEQVDKIFRAAALAAADARIPLAKMAVAESGMGIVEDKVIKNHFASEYIYNAYKDEKTCGVLSEDDTFGTITIAEPIGIICGIVPTTNPTSTAIFKSLISLKTRNAIIFSPHPRAKDATNKAADIVLQAAIAAGAPKDLIGWIDQPSVELSNALMHHPDINLILATGGPGMVKAAYSSGKPAIGVGAGNTPVVIDETADIKRAVASVLMSKTFDNGVICASEQSVVVVDSVYDAVRERFATHGGYLLQGKELKAVQDVILKNGALNAAIVGQPAYKIAELAGFSVPENTKILIGEVTVVDESEPFAHEKLSPTLAMYRAKDFEDAVEKAEKLVAMGGIGHTSCLYTDQDNQPARVSYFGQKMKTARILINTPASQGGIGDLYNFKLAPSLTLGCGSWGGNSISENVGPKHLINKKTVAKRAENMLWHKLPKSIYFRRGSLPIALDEVITDGHKRALIVTDRFLFNNGYADQITSVLKAAGVETEVFFEVEADPTLSIVRKGAELANSFKPDVIIALGGGSPMDAAKIMWVMYEHPETHFEELALRFMDIRKRIYKFPKMGVKAKMIAVTTTSGTGSEVTPFAVVTDDATGQKYPLADYALTPDMAIVDANLVMDMPKSLCAFGGLDAVTHAMEAYVSVLASEFSDGQALQALKLLKEYLPASYHEGSKNPVARERVHSAATIAGIAFANAFLGVCHSMAHKLGSQFHIPHGLANALLICNVIRYNANDNPTKQTAFSQYDRPQARRRYAEIADHLGLSAPGDRTAAKIEKLLAWLETLKAELGIPKSIREAGVQEADFLANVDKLSEDAFDDQCTGANPRYPLISELKQILLDTYYGRDYVEGETAAKKEAAPAKAEKKAKKSA

SEQ ID NO: 7 - protein, 367 aa, Escherichia coli
  MISC_FEATURE (1)..(367): Amino acid sequence of glycerol dehydrogenase gldA from E. coli
MDRIIQSPGKYIQGADVINRLGEYLKPLAERWLVVGDKFVLGFAQSTVEKSFKDAGLVVEIAPFGGECSQNEIDRLRGIAETAQCGAILGIGGGKTLDTAKALAHFMGVPVAIAPTIASTDAPCSALSVIYTDEGEFDRYLLLPNNPNMVIVDTKIVAGAPARLLAAGIGDALATWFEARACSRSGATTMAGGKCTQAALALAELCYNTLLEEGEKAMLAAEQHVVTPALERVIEANTYLSGVGFESGGLAAAHAVHNGLTAIPDAHHYYHGEKVAFGTLTQLVLENAPVEEIETVAALSHAVGLPITLAQLDIKEDVPAKMRIVAEAACAEGETIHNMPGGATPDQVYAALLVADQYGQRFLQEWE

SEQ ID NO: 8 - protein, 584 aa, Saccharomyces cerevisiae
  MISC_FEATURE (1)..(584): Amino acid sequence of dihydroxyacetone kinase DAK1 from S. cerevisiae
MSAKSFEVTDPVNSSLKGFALANPSITLVPEEKILFRKTDSDKIALISGGGSGHEPTHAGFIGKGMLSGAVVGEIFASPSTKQILNAIRLVNENASGVLLIVKNYTGDVLHFGLSAERARALGINCRVAVIGDDVAVGREKGGMVGRRALAGTVLVHKIVGAFAEEYSSKYGLDGTAKVAKIINDNLVTIGSSLDHCKVPGRKFESELNEKQMELGMGIHNEPGVKVLDPIPSTEDLISKYMLPKLLDPNDKDRAFVKFDEDDEVVLLVNNLGGVSNFVISSITSKTTDFLKENYNITPVQTIAGTLMTSFNGNGFSITLLNATKATKALQSDFEEIKSVLDLLNAFTNAPGWPIADFEKTSAPSVNDDLLHNEVTAKAVGTYDFDKFAEWMKSGAEQVIKSEPHITELDNQVGDGDCGYTLVAGVKGITENLDKLSKDSLSQAVAQISDFIEGSMGGTSGGLYSILLSGFSHGLIQVCKSKDEPVTKEIVAKSLGIALDTLYKYTKARKGSSTMIDALEPFVKEFTASKDFNKAVKAAEEGAKSTATFEAKFGRASYVGDSSQVEDPGAVGLCEFLKGVQSAL

SEQ ID NO: 9 - protein, 591 aa, Saccharomyces cerevisiae
  MISC_FEATURE (1)..(591): Amino acid sequence of dihydroxyacetone kinase DAK2 from S. cerevisiae
MSHKQFKSDGNIVTPYLLGLARSNPGLTVIKHDRVVFRTASAPNSGNPPKVSLVSGGGSGHEPTHAGFVGEGALDAIAAGAIFASPSTKQIYSAIKAVESPKGTLIIVKNYTGDIIHFGLAAERAKAAGMKVELVAVGDDVSVGKKKGSLVGRRGLGATVLVHKIAGAAASHGLELAEVAEVAQSVVDNSVTIAASLDHCTVPGHKPEAILGENEYEIGMGIHNESGTYKSSPLPSISELVSQMLPLLLDEDEDRSYVKFEPKEDVVLMVNNMGGMSNLELGYAAEVISEQLIDKYQIVPKRTITGAFITALNGPGFGITLMNASKAGGDILKYFDYPTTASGWNQMYHSAKDWEVLAKGQVPTAPSLKTLRNEKGSGVKADYDTFAKILLAGIAKINEVEPKVTWYDTIAGDGDCGTTLVSGGEALEEAIKNHTLRLEDAALGIEDIAYMVEDSMGGTSGGLYSIYLSALAQGVRDSGDKELTAETFKKASNVALDALYKYTRARPGYRTLIDALQPFVEALKAGKGPRAAAQAAYDGAEKTRKMDALVGRASYVAKEELRKLDSEGGLPDPGAVGLAALLDGFVTAAGY

SEQ ID NO: 10 - protein, 474 aa, Lactobacillus plantarum
  MISC_FEATURE (1)..(474): Amino acid sequence of arabinose isomerase (araA) from Lactobacillus plantarum
MLSVPDYEFWFVTGSQHLYGEEQLKSVAKDAQDIADKLNASGKLPYKVVFKDVMTTAESITNFMKEVNYNDKVAGVITWMHTFSPAKNWIRGTELLQKPLLHLATQYLNNIPYADIDFDYMNLNQSAHGDREYAYINARLQKHNKIVYGYWGDEDVQEQIARWEDVAVAYNESFKVKVARFGDTMRNVAVTEGDKVEAQIKMGWTVDYYGIGDLVEEINKVSDADVDKEYADLESRYEMVQVDNDADTYKHSVRVQLAQYLGIKRFLERGGYTAFTTNFEDLWGMEQLPGLASQLLIRDGYGFGAEGDWKTAALGRVMKIMSHNKQTAFMEDYTLDLRHGHEAILGSHMLEVDPSIASDKPRVEVHPLDIGGKDDPARLVFTGSEGEAIDVTVADFRDGFKMISYAVDANKPEAETPNLPVAKQLWTPKMGLKKGALEWMQAGGGHHTMLSFSLTEEQMEDYATMVGMTKAFLK

SEQ ID NO: 11 - protein, 533 aa, Lactobacillus plantarum
  MISC_FEATURE (1)..(533): Amino acid sequence of L-ribulokinase (araB) from Lactobacillus plantarum
MNLVETAQAIKTGKVSLGIELGSTRIKAVLITDDFNTIASGSYVWENQFVDGTWTYALEDVWTGIQQSYTQLAADVRSKYHMSLKHINAIGISAMMHGYLAFDQQAKLLVPFRTWRNNITGQAADELTELFDFNIPQRWSIAHLYQAILNNEAHVKQVDFITTLAGYVTWKLSGEKVLGIGDASGVFPIDETTDTYNQTMLTKFSQLDKVKPYSWDIRHILPRVLPAGAIAGKLTAAGASLLDQSGTLDAGSVIAPPEGDAGTGMVGTNSVRKRTGNISVGTSAFSMNVLDKPLSKVYRDIDIVMTPDGSPVAMVHVNNCSSDINAWATIFREFAARLGMELKPDRLYETLFLESTRADADAGGLANYSYQSGENITKIQAGRPLFVRTPNSKFSLPNFMLTQLYAAFAPLQLGMDILVNEEHVQTDVMIAQGGLFRTPVIGQQVLANALNIPITVMSTAGEGGPWGMAVLANFACRQTAMNLEDFLDQEVFKEPESMTLSPEPERVAGYREFIQRYQAGLPVEAAAGQAIKY

SEQ ID NO: 12 - protein, 242 aa, Lactobacillus plantarum
  MISC_FEATURE (1)..(242): Amino acid sequence of L-ribulose-5-P-4- epimerase (araD) from Lactobacillus plantarum
MLEALKQEVYEANMQLPKLGLVTFTWGNVSGIDREKGLFVIKPSGVDYGELKPSDLVVVNLQGEVVEGKLNPSSDTPTHTVLYNAFPNIGGIVHTHSPWAVAYAAAQMDVPAMNTTHADTFYGDVPAADALTKEEIEADYEGNTGKTIVKTFQERGLDYEAVPASLVSQHGPFAWGPTPAKAVYNAKVLEVVAEEDYHTAQLTRASSELPQYLLDKHYLRKHGASAYYGQNNAHSKDHAVRK

SEQ ID NO: 13 - protein, 467 aa, Escherichia coli
  MISC_FEATURE (1)..(467): Amino acid sequence of ethanolamine utilizing protein (Ec_eutE) from Escherichia coli
MNQQDIEQVVKAVLLKMQSSDTPSAAVHEMGVFASLDDAVAAAKVAQQGLKSVAMRQLAIAAIREAGEKHARDLAELAVSETGMGRVEDKFAKNVAQARGTPGVECLSPQVLTGDNGLTLIENAPWGVVASVTPSTNPAATVINNAISLIAAGNSVIFAPHPAAKKVSQRAITLLNQAIVAAGGPENLLVTVANPDIETAQRLFKFPGIGLLVVTGGEAVVEAARKHTNKRLIAAGAGNPPVVVDETADLARAAQSIVKGASFDNNIICADEKVLIVVDSVADELMRLMEGQHAVKLTAEQAQQLQPVLLKNIDERGKGTVSRDWVGRDAGKIAAAIGLKVPQETRLLFVETTAEHPFAVTELMMPVLPVVRVANVADAIALAVKLEGGCHHTAAMHSRNIENMNQMANAIDTSIFVKNGPCIAGLGLGGEGWTTMTITTPTGEGVTSARTFVRLRRCVLVDAFRIV

SEQ ID NO: 14 - protein, 669 aa, Saccharomyces cerevisiae
  MISC_FEATURE (1)..(669): Amino acid sequence of FPS1 aquaglyceroporin from Saccharomyces cerevisiae
MSNPQKALNDFLSSESVHTHDSSRKQSNKQSSDEGRSSSQPSHHHSGGTNNNNNNNNNNNNSNNNNNGNDGGNDDDYDYEMQDYRPSPQSARPTPTYVPQYSVESGTAFPIQEVIPSAYINTQDINHKDNGPPSASSNRAFRPRGQTTVSANVLNIEDFYKNADDAHTIPESHLSRRRSRSRATSNAGHSANTGATNGRTTGAQTNMESNESPRNVPIMVKPKTLYQNPQTPTVLPSTYHPINKWSSVKNTYLKEFLAEFMGTMVMIIFGSAVVCQVNVAGKIQQDNFNVALDNLNVTGSSAETIDAMKSLTSLVSSVAGGTFDDVALGWAAAVVMGYFCAGGSAISGAHLNPSITLANLVYRGFPLKKVPYYFAGQLIGAFTGALILFIWYKRVLQEAYSDWWMNESVAGMFCVFPKPYLSSGRQFFSEFLCGAMLQAGTFALTDPYTCLSSDVFPLMMFILIFIINASMAYQTGTAMNLARDLGPRLALYAVGFDHKMLWVHHHHFFWVPMVGPFIGALMGGLVYDVCIYQGHESPVNWSLPVYKEMIMRAWFRRPGWKKRNRARRTSDLSDFSYNNDDDEEFGERMALQKTKTKSSISDNENEAGEKKVQFKSVQRGKRTFGGIPTILEEEDSIETASLGATTTDSIGLSDTSSEDSHYGNAKKVT